(19) United States
(12) Patent Application Publication
(10) Pub. No.: US 2022/0213144 A1
(43) Pub. Date: Jul. 7, 2022

(54) HALOGENATED ANTIMICROBIAL PEPTOIDS

(71) Applicant: Maxwell Biosciences, Inc., Westlake Hills, TX (US)

(72) Inventors: NATALIA MOLCHANOVA, K0benhavn V (DK); ANNELISE BARRON, Redwood City, CA (US); HAVARD JENSSEN, Roskilde (DK)

(21) Appl. No.: 17/606,761

(22) PCT Filed: Apr. 30, 2020
(86) PCT No.: PCT/US20/30890

§ 371 Date: Oct. 27, 2021

Related U.S. Application Data
(60) Provisional application No. 62/841,227, filed on Apr. 30, 2019.

(51) Int. Cl. C07K 7/08; A61P 31/04

(52) U.S. Cl. CPC C07K 7/08; A61K 38/00; A61P 31/04

(57) ABSTRACT

A poly-N-substituted glycine compound of a formula

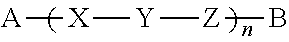

is provided, wherein A is a terminal N-alkyl substituted glycine residue; n is an integer; B is selected from the group consisting of NH₂, one and two N-substituted glycine residues, and wherein said one and two N-substituted glycine residues have N-substituents which are independently selected from natural α-amino acid side chain moieties, isomers and carbon homologs thereof, X, Y and Z are independently selected from the group consisting of N-substituted glycine residues, wherein said N-substituents are independently selected from the group consisting of natural α-amino acid side chain moieties, isomers and carbon homologs thereof, and proline residues, and wherein at least one of A, B, X, Y and Z contains a halogen-bearing moiety.

### CROSS-REFERENCE TO RELATED APPLICATION

The present application is a national stage filing of PCT/US20/30890, filed on Apr. 30, 2020, which has the same title and the same inventors, and which is incorporated herein by reference in its entirety; which claims priority to U.S. Provisional Application No. 62/841,227, filed Apr. 30, 2019, having the same title, and having the same inventors, and which is incorporated herein by reference in its entirety.

### FIELD OF THE DISCLOSURE

The present disclosure relates generally to antimicrobial compositions, and more particularly to antimicrobial peptoids.

### BACKGROUND OF THE DISCLOSURE

The use of natural antimicrobial peptides (AMPs) in the treatment of multi-drug resistant bacteria has been the focus of a considerable amount of research. AMPs are known to defend a wide array of organisms against bacterial pathogens. These peptides have shown potential as supplements for (or replacements of) conventional antibiotics, since few bacteria have evolved resistance to them.

AMPs destroy bacteria in various ways. Some AMPs kill bacteria by permeating the cytoplasmic membrane and causing depolarization or leakage of internal cell materials. Other AMPs function by targeting anionic bacterial constituents, such as DNA, RNA, or cell wall components. Bacterial resistance to AMPs is rare, possibly because such AMPs have evolved along with the resistance mechanisms that are designed to evade them. When bacteria do show resistance to certain AMPs, via the production of so-called “virulence factors”, these virulence factors are molecules that bind to and inactivate those certain human AMPs. However, generally, the targets of many AMPs (such as bacterial plasma membranes and anionic intracellular macromolecules) are sufficiently general that changes to the sequence of the AMP can be made to subvert resistance, without having any significant adverse impact on the overall functionality of the AMP.

Although AMPs have been actively studied for decades, only a few particular AMPs have achieved widespread clinical use (for instance: Colistin, Polymyxin E). This slow clinical adoption of AMPs has been due, in part, to the vulnerability of many peptide therapeutics to rapid in vivo degradation (and in particular, enzymatic and proteolytic degradation), which dramatically reduces their bioavailability. This requires large doses, which greatly increases expense.

### SUMMARY OF THE DISCLOSURE

In one aspect, a poly-N-substituted glycine compound of a formula

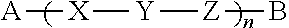

or a pharmaceutically acceptable salt thereof is provided wherein

A is a terminal N-alkyl substituted glycine residue;

n is an integer;

B is selected from the group consisting of NH₂, one and two N-substituted glycine residues, and wherein said one and two N-substituted glycine residues have N-substituents which are independently selected from natural α-amino acid side chain moieties, isomers and carbon homologs thereof; and

X, Y and Z are independently selected from the group consisting of N-substituted glycine residues, wherein said N-substituents are independently selected from the group consisting of natural α-amino acid side chain moieties, isomers and carbon homologs thereof, and proline residues, and wherein at least one of A, B, X, Y and Z contains a halogen-bearing moiety.

In another aspect, a poly-N-alkyl substituted glycine compound of a formula

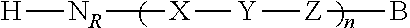

or a pharmaceutically acceptable salt thereof is provided wherein

B is selected from NH₂ and X′;

N_{R}, X, Y, Z and X′ are independently selected from N-substituted glycine residues containing N-substituents, wherein said N-substituents of said N-substituted glycine residues are independently selected from natural α-amino acid side chain moieties, isomers and carbon homologs thereof, and proline residues, and wherein at least one said N-substituent contains a halogen atom;

n is an integer; and

R is an N-alkyl substituent of said N_{R} glycine residue, said substituent selected from about C₄ to about C₂₀ linear, branched and cyclic alkyl moieties.

In a further aspect, a poly-N-substituted glycine or a pharmaceutically acceptable salt thereof is provided comprising an N-terminal N-alkyl substituted glycine residue, where said alkyl substituent is selected from about C₄ to about C₂₀ linear, branched and cyclic alkyl moieties; a C-terminus selected from NH₂, one and two N-substituted glycine residues, said N-substituents independently selected from α-amino acid side chain moieties and carbon homologs thereof, and 2 to about 15 monomeric residues between said N- and C-termini, each said residue independently selected from proline residues and N-substituted glycine residues, said N-substituents independently selected from natural α-amino acid side chain moieties, isomers and carbon homologs thereof, at least one said monomeric residue is N_{Lys} and at least one said N-substituent is chiral, said monomeric residues selected to provide said compound a periodic or non-periodic sequence of monomeric residues; and wherein at least one of said residues contains at least one halogen substituent.

In still another aspect, a compound, or a pharmaceutically acceptable salt thereof, is provided which is derived from a material selected from the group consisting of the compound of FIG. 1 and the compound of FIG. 13 by substituting at least one hydrogen atom in at least one aryl moiety thereof with at least one halogen atom.

In yet another aspect, a method is provided for treating or inhibiting a disease, comprising administering to an individual who has, or is at risk of developing said disease, an amount of at least one poly-N-alkyl substituted glycine or a pharmaceutically acceptable salt thereof, wherein the amount of the poly-N-alkyl substituted glycine is effective to treat or inhibit said disease, and wherein the poly-N-alkyl substituted glycine compound has the formula

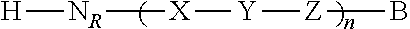

wherein

B is selected from NH₂ and X′;

N_{R}, X, Y, Z and X′ are independently selected from N-substituted glycine residues containing N-substituents, wherein said N-substituents of said N-substituted glycine residues are independently selected from natural α-amino acid side chain moieties, isomers and carbon homologs thereof, and proline residues, and wherein at least one said N-substituent contains a halogen atom;

n is an integer; and

R is an N-alkyl substituent of said N_{R} glycine residue, said substituent selected from about C₄ to about C₂₀ linear, branched and cyclic alkyl moieties.

In a further aspect, a method is provided for treating or inhibiting a disease, comprising administering to an individual who has, or is at risk of developing said disease, an amount of at least one poly-N-alkyl substituted glycine or a pharmaceutically acceptable salt thereof, wherein the amount of the poly-N-alkyl substituted glycine is effective to treat or inhibit said disease, and wherein the poly-N-alkyl substituted glycine compound is a compound, or a pharmaceutically acceptable salt thereof, which is derived from a material selected from the group consisting of the compound of FIG. 1 and the compound of FIG. 13 by substituting at least one hydrogen atom in at least one aryl moiety thereof with at least one halogen atom.

In still another aspect, a poly-N-substituted glycine compound is provided which contains at least one halogen selected from the group consisting of chlorine, bromine and iodine. The poly-N-substituted glycine compound preferably contains a carboxamide terminus group, and is preferably made via a rink amide resin.

### BRIEF DESCRIPTION OF THE DRAWINGS

FIG. 1 depicts the molecular structures of first-generation peptoids containing halogen atoms. The different peptoid oligomer structures are numbered 1-37, and 38-51.

FIG. 2 is a set of graphs of Small-Angle X-Ray Scattering (SAXS) data showing the scattered intensity plotted towards the modulus of the scattering vector Q for 10mer peptoids at 5 mg/ml and 37° C. obtained at the BM29 beam line, at the ESRF laboratory. FIG. 2A displays results for the fully halogenated peptoids, while FIG. 2B displays results for the half-halogenated peptoids. The non-halogenated peptoid (compound 19) is included in both graphs as a reference. Results indicate predominantly free unstructured peptoid chains with a small fraction of sheet-like filaments for compounds 19-21 and 24-27, and defined bundles for compounds 22 and 23 (seen as an increase in the scattering intensity and a change in shape of the curve).

FIG. 3 depicts full SAXS results for the fully iodinated peptoids measured with Bruker NANOSTAR instrumentation (except the 10-mer compound 23, which was measured at ESRF) in the indicated concentrations together with model fits (fitted using the bundle model as described herein). FIGS. 3A, 3B, 3C and 3D shows the 6-mer (compound 5), 8-mer (compound 14), 10-mer (compound 23) and 12-mer (compound 32), respectively.

FIG. 4 depicts the chlorinated and brominated variants of Peptoid 1 [H-(NLys-Nspe-Nspe)₄-NH₂], and shortened brominated Peptoid 1 analogues.

FIG. 5 is a graph of IC50 curves of selected peptoids toward an HaCaT cell line.

FIG. 6 depicts the structure of Peptoid 1 [H-(NLys-Nspe-Nspe)₄-NH₂], where NLys is N-(4-aminobutyl)glycine and Nspe is N—(S)-(1-phenylethyl)glycine.

FIGS. 7-12 depict the structures of several particular, non-limiting examples of halogenated analogs of Peptoid 1.

FIG. 13 depicts the structure of a shorter, modified version of Peptoid 1, which rather than being composed of 12 monomers, comprises 6 monomers.

FIGS. 14-16 depict the structures of some particular, non-limiting examples of halogenated analogs of the peptoid whose structure is depicted in FIG. 17.

FIG. 17 is a graph depicting the cyctotoxicity (HaCaT cell line) of some of the peptoids disclosed herein.

FIG. 18 depicts SAXS results for all 10-mers measured at ESRF in the indicated concentrations together with model fits (the data for compounds 19-21 and 24-27 was fitted using a model for Random polymer-like chains with fiber-like clusters model (EQUATION 1), while the data for compounds 22 and 23 was fitted using the bundle model (EQUATION 6)).

### DETAILED DESCRIPTION

### I. Background

The foregoing problems have led to the development of peptidomimetics, which are small, protein-like chains designed to mimic a peptide. Peptidomimetics may be made by modifying an existing peptide. Peptidomimetics may also be based on similar systems that mimic peptides, such as peptoids and β-peptides.

Peptoids (oligomers of N-substituted glycines) are isomers of peptides in which side chains are attached to the backbone amide nitrogen rather than to the α-carbon. Antimicrobial peptoids have been described, for example, in U.S. Pat. No. 8,445,632 (Barron et al.), entitled “Selective Poly-N-Substituted Glycine Antibiotics”, which is incorporated herein by reference in its entirety. Peptoids demonstrate proteolytic stability and better bioavailability than corresponding peptides, while in many cases retaining antibacterial activity.

Peptoids are particularly well-suited for AMP mimicry. Peptoids are easily synthesized using conventional peptide synthesis equipment, and provide access to diverse sequences at relatively low cost. Submonomer synthetic methods are known that may be utilized to impart a wide variety of chemical functionalities to peptoids. Consequently, peptoids are highly and finely tunable. Furthermore, they are protease-resistant, and can be designed to form amphipathic helices that resist thermal and chaotropic denaturation.

Despite their promise, further improvements in peptoids are needed in order for these materials to achieve their full potential as antimicrobial therapeutics. In particular, there is a need in the art for further improvements in the antimicrobial efficacy of peptoids without a concomitant increase in cytotoxicity. Similarly, there is a need in the art for further a means for reducing the cytotoxicity of existing peptoids without a concomitant decrease in the antimicrobial efficacy of these materials. There is also a need in the art for peptidomimetics having novel substituents which may increase the efficacy of existing peptoids materials against particular pathogens. There is further a need in the art for a means to manipulate the ability of peptoids to form aggregates, which may be implicated in the pharmaceutical efficacy of these materials.

The need for these improvements has been underscored by the well documented increase in the resistance of many pathogens to current treatments. By way of example, in recent years, methicillin-resistant S. pseudintermedius (MRSP) strains have emerged worldwide. Methicillin-resistant S. pseudintermedius is resistant to all 3-lactam antibiotics. Multidrug-resistant (MDR) strains of this and other pathogens have emerged as well, which display resistance to virtually all antimicrobial agents that are currently available. The most recent example is the novel strain of coronavirus that has resulted in the current COVID-19 pandemic.

Recently, it was shown that the incorporation of fluorine atoms into certain peptidomimetics can improve the antimicrobial activity of these compositions, while leaving their haemolytic activity unaffected. See Molchanova N, Hansen P R, Damborg P, Franzyk H., “Fluorinated antimicrobial lysine-based peptidomimetics with activity against methicillin-resistant Staphylococcus pseudintermedius”, J Pep Sci. 2018; e3098 (Molchanova et al.). In particular, fluorination of certain peptidomimetics was found to have a pronounced effect on the activity of these materials against Gram-positive bacteria. However, the effect of introducing other halogens into antimicrobial peptoids has been poorly explored.

The present investigators have addressed this issue by synthesizing a library of halogenated peptoids. These peptoids contain one or more fluorine, chlorine, bromine and/or iodine atoms, and vary by length and level of halogen substitution in position 4 of the phenyl rings. A clear correlation was observed in these materials between halogenation of an inactive model peptoid and its increased antimicrobial activity.

Consequently, chlorinated and brominated analogues of some known peptoids and their shorter counterparts were produced. The shorter brominated analogues displayed notable improvements (up to 32-fold) in activity against S. aureus, and similarly displayed significant improvements (16- to 64-fold) in activity against E. coli and P. aeruginosa. In many cases, these improvements in efficacy were achieved while also achieving reductions in cytotoxicity.

Without wishing to be bound by theory, it is believed that the biological effect of halogen substitution is linked to the relative hydrophobicity and self-assembly properties of the resulting compounds. Small angle X-ray scattering (SAXS) suggests that the self-assembled structures are dependent on (a) the size of the halogen, (b) the degree of substitution in the halogenated peptoid, and (c) the length of the peptoid. As described in greater detail below, these features have been correlated to the activity of these peptoids.

### II. Overview

The rapid emergence and widespread distribution of antibacterial resistance is recognized as one of the most serious global threats to human health. [1-4]. Most antibiotics in clinical use are becoming less effective in treating instances of infections caused by Gram-positive or Gram-negative superbugs. [5-6] Consequently, there is an increasing need in the art for new antibiotics or alternative therapeutics that may be utilized for treating these pathogens.

Antimicrobial peptides (AMPs) are an essential part of the innate immune system of nearly all living organisms, and are still considered a promising therapeutic strategy in fighting bacterial infections. [7] AMPs have attracted more attention recently, with the rise of antimicrobial resistance in many pathogens and the concomitant need for new antibiotics. As a result, 36 different AMPs are currently undergoing clinical trials against various infectious diseases. [8] However, the practical application of AMPs is limited by the rapid in vivo degradation of these materials and by issues with systemic toxicity. Currently, the production costs of these materials is also prohibitive. [9]

Poly-N-substituted glycines (peptoids) are a class of peptidomimetics in which the side chains are attached to the backbone amide nitrogen rather than to the α-carbon. [10] Antimicrobial peptoids were first developed in 2003 (see Patch J A, Barron A E, “Helical peptoid mimics of magainin-2 amide,” Journal of the American Chemical Society 2003, 125: 12092-12093). Over the past two decades, various antimicrobial peptoids have been produced. Many of these materials have been found to maintain their stability and antimicrobial activity in vivo. [11]

Over 4000 halogenated compounds have been isolated from natural sources. This diverse group of natural products display a wide range of biological activities, including anticancer and antimicrobial properties. [12-13] However, until recently, limited attention has been given to ascertaining the antimicrobial properties of peptides and peptidomimetics containing halogen atoms. To date, fluorination has received the most interest, although studies investigating the link between fluorination and antimicrobial activity have yielded inconclusive results.

For example, the introduction of hexafluoroleucine into magainin and buforin was found to confer enhanced antimicrobial activity to these materials, while retaining their low hemolytic properties. Similarly, the presence of fluorine atoms and trifluoromethyl groups was found to improve the potency of short cationic peptides. [14-16] By contrast, the incorporation of hexafluoroleucine into protegrin analogs led to decreased potency of these analogs, while lipopeptide variants with fluorinated tails demonstrated only moderate antibacterial activity and pronounced hemolytic properties. [17-18]

Recently, Molchanova et. al. reported a link between the introduction of fluorine atoms into peptidomimetic sequences and increased antimicrobial activity of these materials against Gram-positive bacteria. This increase in antimicrobial activity was achieved without adversely affecting hemolytic properties. [19-20] Similarly, both vancomycin and salinosporamid A have been found to require the presence of one to two chlorine substituents to achieve their antimicrobial activity. [21-22]

Jia et. al. found that the introduction of fluorine, chlorine, bromine and iodine into the honeybee peptide Jelleine-1 (via halogen-substituted phenylalanine) led to improved protease stability. One of the fluorinated analogues showed antimicrobial activity similar to that of the parent peptide, while the chlorinated, brominated and iodinated analogues displayed a 2-8 fold increase in activities in vitro. [23] Interestingly, the in vitro antimicrobial activity of the iodine analogue was the highest, although the chlorinated and brominated versions displayed a more potent efficacy in vivo.

Both chlorinated and brominated variants of antibiotic NAI-107 have demonstrated higher antimicrobial activity, with the brominated variant displaying slightly higher potency. [24]

Dodecamer Peptoid 1 [H-(NLys-Nspe-Nspe)₄-NH₂] is an example of a well-studied, promising antimicrobial peptoid with a wide spectrum of antimicrobial activity, although it exhibits relatively high cytotoxicity in vitro (it is to be noted, however, that Peptoid 1 has been tested and found to be reasonably well tolerated intraperitoneally in vivo against Staph. aureus). See Czyzewski et al. (2016) In Vivo, In Vitro, and In Silico Characterization of Peptoids as Antimicrobial Agents. PLoS ONE, 11 (2): e0135961. doi:10.1371/journal.pone.0135961. PMCID: 26849681. Recent attempts to enhance the antimicrobial potency of Peptoid 1 by varying its structure have also been reported. However, the incorporation of fluorine or chlorine atoms into the Nspe units of Peptoid 1 was not found to lead to significant improvements in the antimicrobial profile of this compound. [25]

The introduction of halogens into the chemical structure of peptides or peptoids is known to generally increase the hydrophobicity of these molecules. [26] This may lead to conformational changes and self-assembly into supramolecular nanostructures, driven by increased hydrophobic interactions. The correlation between antimicrobial activity and self-assembly has been extensively discussed in the literature, where the impact on antimicrobial properties and overall toxicity trends in both directions. [27-28] For example, Xu and co-workers found a link between increased antimicrobial activity and the self-assembly of defined supramolecular nanofibers. On the other hand, Chu-Kung and co-workers found a clear tendency for their fatty acid conjugated peptides to show reduced antimicrobial activity. [29-30] Antimicrobial peptoids are significantly less prone to fold and form secondary structures, and there do not appear to have been any studies directed to the effect of self-assembly on the antimicrobial efficacy of peptoids.

A structure-activity investigation of halogenated peptoids is described below. The aim of this investigation was to ascertain the effect of the nature of the halogen and the amount of halogen substitution on (a) the ability of the halogenated peptoids to self-assemble into nanostructures, and (b) the antimicrobial activity of the halogenated peptoids. The incorporation of halogen atoms into the scaffold of Peptoid 1 and its repeating sequence elements was also investigated in an attempt to increase the antimicrobial activity, or to modulate the cytotoxicity, of this peptoid.

### III. Results and Discussion

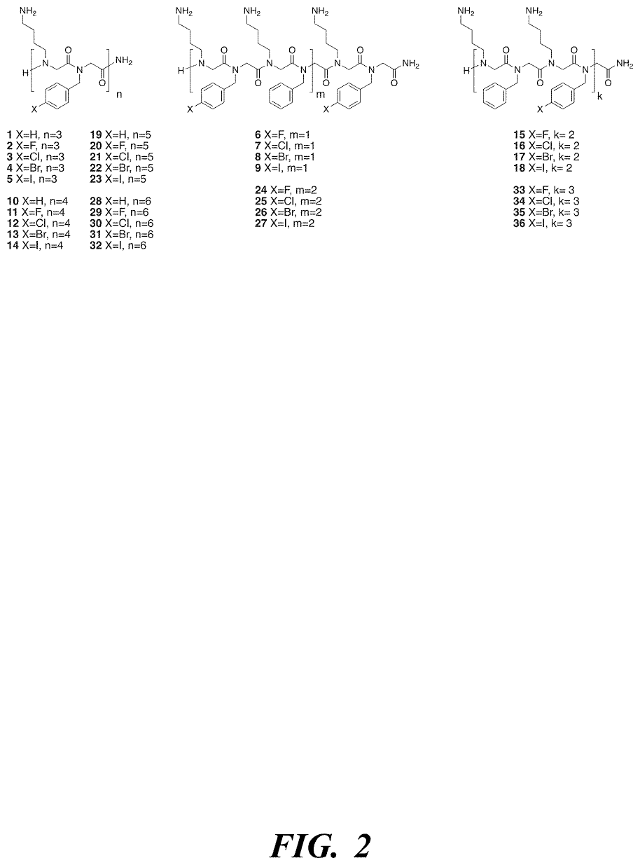

A set of 36 peptoids was synthesized using a scaffold containing alternating NLys and Npm units which varied by length (6-, 8-, 10-, 12-mers), and in the level of halogen substitution (full or alternate). Halogen atoms (fluorine, chlorine, bromine or iodine) were introduced via phenyl rings in position 4 and were synthesized using a submonomer approach (see FIG. 2).

All 36 peptoids were tested against seven bacterial strains. These included five Gram-positive strains (Staphylococcus aureus ATCC 25923 and ATCC 29213, methicillin-resistant Staphylococcus aureus USA 300, methicillin-restant Staphylococcus epidermidis ET-024 and ATCC 51625) and two Gram-negative strains (Escherichia coli ATCC 25922 and Pseudomonas aeruginosa PA01) (see TABLE 1). The non-halogenated peptoids demonstrated no potency against the selected bacterial strains. Thus, compounds 1, 10, 19 and 28 (a 6mer, an 8mer, a 10mer, and a 12mer, respectively) represent the inactive controls.

TABLE 1
Minimal inhibitory concentrations (μg/mL) of the first generation
peptoids containing halogen atoms.

| | RP- | | | | | | | |
|---|---|---|---|---|---|---|---|---|
| cmpd | HPLC RT | SA^{a} | SA^{b} | MRSA^{c} | MRSE^{d} | MRSE^{e} | EC^{f} | PA^{g} |
| 1 | 11.49 | >512 | >512 | >512 | 512 | 512 | >512 | >512 |
| 2 | 12.08 | >512 | >512 | 512 | 128 | 512 | 512 | >512 |
| 3 | 13.06 | 64 | 64 | 16 | 16 | 8 | 512 | 512 |
| 4 | 13.32 | 16 | 16 | 16 | 4 | 4 | 64 | >512 |
| 5 | 13.67 | 8 | 4 | 4 | 4 | 2 | 64 | >512 |
| 6 | 12.00 | >512 | >512 | >512 | 256 | 512 | >512 | >512 |
| 7 | 12.53 | 256 | 256 | 128 | 64 | 32 | 256 | 512 |
| 8 | 12.72 | 128 | 64 | 64 | 32 | 16 | 256 | 256 |
| 9 | 12.97 | 64 | 16 | 16 | 16 | 4 | 256 | 256 |
| 10 | 11.69 | >512 | >512 | >512 | 256 | 256 | 256 | >512 |
| 11 | 12.30 | 256 | 256 | 256 | 64 | 32 | 128 | >512 |
| 12 | 13.32 | 4 | 4-8 | 2 | 2 | 1 | 64 | >512 |
| 13 | 13.60 | 4 | 8 | 2 | 2 | 4 | 64 | >512 |
| 14 | 13.94 | 8 | 16 | 4 | 2 | 4 | 64 | >512 |
| 15 | 12.01 | >512 | >512 | >512 | 128 | 128 | 512 | 512 |
| 16 | 12.55 | 16 | 64 | 16 | 8 | 8 | 256 | 512 |
| 17 | 12.67 | 16 | 32 | 32 | 4 | 4 | 256 | 512 |
| 18 | 12.94 | 32 | 32 | 16 | 8 | 4 | 256 | >512 |
| 19 | 11.84 | >512 | >512 | >512 | 128 | 128 | 256 | 512 |
| 20 | 12.41 | 64 | 64 | 32 | 4 | 8 | 128 | 256 |
| 21 | 12.96 | 2 | 2 | 2 | 1 | 2 | 64 | >512 |
| 22 | 13.75 | 2 | 2 | 4 | 1 | 1 | 64 | >512 |
| 23 | 14.09 | 32 | 32 | 16 | 4 | 8 | >512 | >512 |
| 24 | 12.20 | 512 | 512 | 256 | 4 | 8 | 256 | 512 |
| 25 | 12.85 | 8 | 8 | 16 | 2 | 1 | 256 | 512 |
| 26 | 13.02 | 4 | 4 | 8 | 1 | 1 | 256 | 512 |
| 27 | 13.25 | 4 | 4 | 4 | 2 | 1 | 128 | >512 |
| 28 | 11.93 | >512 | 512 | 512 | 64 | 32 | 128 | 128 |
| 29 | 12.52 | 32 | 32 | 16 | 4-8 | 4 | 256 | 64 |
| 30 | 13.57 | 2 | 2 | 2 | 2 | 1 | 64 | >512 |
| 31 | 13.85 | 4 | 8 | 2 | 4 | 4 | 64 | 256 |
| 32 | 14.16 | 64 | 128 | 32 | 32 | 16 | >512 | >512 |
| 33 | 12.25 | 256 | 256 | 128 | 16 | 4 | 256 | 256 |
| 34 | 12.43 | 16 | 8 | 8 | 4 | 1 | 256 | >512 |
| 35 | 12.95 | 8 | 8 | 8 | 8 | 1 | 256 | >512 |
| 36 | 13.16 | 8 | 16 | 4 | 4 | 1 | 256 | >512 |

^{a}S. aureus ATCC 25923;
^{b}S. aureus ATCC 29213;
^{c}methicillin-resistant S. aureus USA 300;
^{d}methicillin-restant S. epidermidis ET-024;
^{e}methicillin-restant S. epidermidis ATCC 51625;
^{f}E. coli ATCC 25922;
^{g}P. aeruginosa PA01;
RP-HPLC RT-reversed-phase HPLC retention time.

The 36 compounds are divided in four sets according to their length. Each set contains a non-substituted control, four fully substituted peptoids with fluorine, chlorine, bromine or iodine, and four “half substituted” peptoids where every second phenyl ring is substituted with a halogen atom.

Halogenation was found to have no effect on the activity of these peptoids against either E. coli or P. aeruginosa (see TABLE 1). However, across all sets, a clear correlation was observed between the antimicrobial activity against Gram-positive strains, the level of substitution, and the nature of a halogen. The fully halogenated peptoids demonstrated drastically enhanced activity against wild type and resistant strains of both S. aureus and S. epidermidis. Interestingly, activity in the 6-mers (compounds 2-5) and 8-mers (compounds 11-14) rose from fluorine to iodine, with the latter being the most potent. For the 6-mers, addition of just three iodine atoms yielded an increase in activity of more than 64-fold against S. aureus (MIC for 1=>512 μg/mL; for 5=8 g/mL) and a 256-fold increase in activity against MRSE (MIC for 1=512 μg/mL; for 5=2 μg/mL), while 8-mer compound 14 exhibited the same activity against S. aureus, and a 128-fold increase in activity against MRSE (MIC for 10=256 μg/mL; for 14=2 g/mL). However, in progressing to fully substituted 10-mer (compounds 20-23) and 12-mer (compounds 29-32) sets, the antimicrobial trend is lost; in particular, compounds bearing iodine atoms displayed lower potency against both S. aureus and multidrug resistant S. epidermidis, as compared to their chlorinated and brominated analogues. Compared to the unsubstituted control peptoids, bromination of the 10-mer led to a greater than >256-fold increase in activity against S. aureus, and a 128-fold increase in activity against S. aureus (S. aureus: MIC for 19=>512 μg/mL; for 22=2 μg/mL; MRSE: MIC for 19=128 μg/mL; for 22=1 μg/mL). By contrast, a brominated 12-mer analogue displayed similar or lower activity against both S. aureus and S. epidermidis.

The “half-substituted” sets fell under a similar trend, though generally displaying similar or lower potency. Interestingly, the half-substituted peptoids bearing bromine exhibited comparable activity to their fully substituted analogues. For example, compound 26 showed MICs between 1-8 μg/mL versus 1-4 μg/mL for the fully substituted analogue (compound 22).

As expected, hydrophobicity of the peptoids increased with the addition of halogen atoms, where fluorine displayed a less pronounced effect while incorporation of iodine led to noticeably higher hydrophobicity profiles. In parallel, the antimicrobial activity of the peptoids fell into a well-established correlation, where increased hydrophobicity led to increased antimicrobial activity. However, when a certain hydrophobicity threshold was met (e.g., for compounds 23 and 32), the activity was lost. This phenomenon has previously been observed in other peptide/peptoid studies. [31-32] Overall, it was found that introduction of halogen atoms led to an increase in hydrophobicity and an increase in antimicrobial activity against Gram-positive bacteria. However, once a certain threshold of hydrophobicity is surpassed, the activity of the peptoids begins to diminish.

### VI. Materials and Methodologies

The following materials and methodologies were utilized in the examples set forth herein.

### 1. General Information

Starting materials and solvents were purchased from commercial suppliers (Iris Biotech, Sigma-Aldrich, and Merck) and used without further purification. Water used for analytical and preparative high-performance liquid chromatography (HPLC) was filtered through a 0.22-μm Millipore membrane filter.

For compounds 1-48, purity was determined by analytical HPLC using a Waters 717 plus Autosampler, In-line Degasser AF, 600 Controller, and 2996 Photodiode Array Detector; the column used was a Waters Symmetry C18, 5 μm, 4.6 mm×250 mm. An aqueous acetonitrile (MeCN) gradient with 0.1% trifluoroacetic acid (TFA) added (eluent A: 5:95 MeCN-H₂O+0.1% TFA, eluent B: 95:5 MeCN-H₂O+0.1% TFA) was employed. All tested compounds had a purity of at least 95%. Preparative HPLC was performed by using a Waters XSelect Peptide CSH C18 OBD™, 5 μm, 19 mm×250 mm column and the same eluents as for analytical HPLC. High-resolution mass spectrometry (HRMS) spectra were obtained by using a Waters QTOF premier mass spectrometer equipped with an electrospray ionization source and a Quadruple and time of flight MS detector.

For compounds 49-51, pep1-6mer and Peptoid 1, product purity was determined by means of analytical UPLC/MS using a Water Acquity UPLC system, equipped with an Acquity Diode Array UV detector and a Waters SQD2 mass spectrometer. As stationary phase, a Waters Acquity UPLC Peptide BEH C18 Column (300 Å pore size, 1.7 μm particle size, 2.1 mm×100 mm) with an Acquity UPLC BEH C18 VanGuard pre-column (1.7 μm, 2.1 mm×5 mm) was employed. Purification by means of preparative HPLC was carried out using a Waters Prep150LC system, equipped with a Waters 2489 UV/Visable detector and a Waters Fraction Collector III collector. As the stationary phase, a Waters XBridge BEH300 Prep C18 column (5 m particle size, 19 mm×100 mm) with a Waters XBridge Peptide BEH300 C18 guard column (5 m particle size, 19 mm×10 mm) was utilized.

### 2. General Synthesis of Peptoids

All peptoids were synthesized manually on Rink amide resin (Novabiochem, 0.65 mmol/g) according to the submonomer method. [10] After synthesis, oligomers were cleaved and deprotected in trifluoroacetic acid (TFA)/triisopropylsilane/water (95:2.5:2.5 by vol.) for 30 min.

### 3. Determination of Minimal Inhibitory Concentration

Bacterial growth inhibition was determined by using broth microdilution according to the Clinical Laboratory Standards Institute. [37] The antibacterial activity of peptoids was tested against S. aureus ATCC 25923, ATCC29213, and methicillin resistant S. aureus USA 300, methicillin resistant S. epidermidis ATCC 51625, a biofilm producing methicillin resistant S. epidermidis ET-02438, P. aeruginosa Pa01 (H103), and E. coli ATCC 25922. Bacteria, grown on agar plates for 18 hours at 37° C., were diluted to ˜1×108 CFU/mL in Mueller-Hinton Broth II (MHB II). Twofold serial dilutions of peptoids in MHB II were inoculated with the bacteria to achieve a final concentration of 5×105 CFU/mL in polypropylene 96 U-well microtiter plates (Corning™ 3897; ThermoFisher Scientific, Roskilde), followed by incubation at 37° C. in ambient air for 18 hours. The MIC values were determined as the lowest concentration showing no visible bacterial growth. Experiments were performed twice (in technical triplicates) on different days.

### 4. Small Angle X-Ray Scattering

SAXS experiments of the 10-mer peptoids were performed on the automated BM29 bioSAXS beamline at the European Synchrotron Radiation Facility (ESRF) in Grenoble, France. [39] The data was obtained using an energy of 12.5 keV and a detector distance of 2.87 m, covering a q range (q=4π sin(θ/2)/λ), where θ is the scattering angle and λ is the X-ray wavelength) of about 0.0047 Å-1 to 0.5 Å-1. The data set was calibrated to an absolute intensity scale using water as a primary standard. Samples (40 μL) were run through a capillary using the flow mode of the automated sample changer. [40] SAXS data was collected in ten successive frames of 0.5 s each to monitor radiation damage, and the data reduction was done using the standard tool at BM29. [41]

The SAXS experiments on the fully iodinated 6-, 8-, and 12-mers to determine the CAC of the compounds were performed using a Bruker NANOSTAR equipped with a microfocus X-ray source (IμS Cu, Incoatec, Germany) and a VANTEC-2000 detector. Raw scattering data was calibrated to absolute intensity scale using water as a primary standard and radially averaged in order to obtain the 1D scattered intensity profile as a function of the scattering vector, with a wavelength of 1.54 Å. Two concentrations of compound 23 and compound 19 were also run on the NANOSTAR to verify that the results were comparable with the results from synchrotron SAXS at ESRF.

### 5. Cell Culturing

An immortalized human keratinocytes (HaCaT) cell line (Gift from David Gram Naym at Bispebjerg Hospital) was cultured to ˜90% confluence after 21-25 h of growth under standard conditions (5% CO₂/95% 02 at 37° C.). Cells were cultured in Dulbecco's Modified Eagle's Medium supplemented with 10% (v/v) fetal bovine serum (FBS). All culture media were supplemented with penicillin (100 IU/mL) and streptomycin (100 μg/mL). All cell media and supplements were obtained from Sigma-Aldrich (St. Louis, Mo., United States). The 96-well plates were obtained from Corning Costar (Sigma-Aldrich, Brondby, Denmark).

### 6. Cell Viability Assay

Cell viability assessment was performed on cell monolayers grown to ˜90% confluence in 96-well plates using the MTS/PMS assay as previously described. [42] Briefly, the adhered cells were washed with 37° C. PBS solution (ThermoFisher Scientific, Roskilde) and exposed for 1 h at 37° C. to 100 μL of peptoid dissolved in the medium also used for culturing of the cell line (at concentrations in the range 0-1000 μg/mL). The cells were then washed twice with 37° C. PBS. A 100 μL aliquot of an MTS/PMS solution in media (consisting of 240 μg/mL MTS (Promega, Madison, Wis., United States) and 2.4 mg/mL PMS (Promega, Madison, Wis., United States)) was added to the cells, which then were incubated for 1 h at 37° C. while being protected from light. A plate reader (SpectraMax i3X; Molecular devices, San Jose, Calif.) was used to measure the absorbance at 492 nm. The relative viability was calculated by using 0.2% (w/v) sodium dodecyl sulfate (SDS) as the positive control, while cells exposed to medium without test compound were used as the negative control. Data was obtained in three independent biological replicates which were performed on separate passages of cells and on separate days, for a total of six replicates.

### 7. Theoretical Modelling Of Data From SAXS

a. Random Polymer-Like Chains with Fiber-Like Clusters

In order to extract accurate and detailed structural information, the SAXS data for the pure peptoids chains were analysed using a combination of free chains and rectangular fibres characterized by dimensions a<b<c, where c is the length of the fibers and a and b is the X and Y direction of the cross section, using EQUATION 1 below:

I(q)=ϕ·Vₚ·Δρ²·(P_{chain}(q)·f_{chain}+Nₚ·Pₛₕₑₑₜ(q)·(1−f_{chain})) (EQUATION 1)

[43]. Here, ϕ is the volume fraction of the polymer, Vₚ is the volume of the polymer, Δρ is the excess scattering length density and f_{chain} is the fraction of free chains. Nₚ, the average number of peptides in each sheet, is defined as

$${N}_{p} = \frac{abc}{{V}_{p}} \cdot {P}_{chain} ( q )$$

is the form factor of the free peptoid chains given by the Debye expression for Gaussian chains:

$${P}_{chain} ( q ) = \frac{2 \cdot exp [ - {( {qR}_{g} )}^{2} ] - 1 + {( {qR}_{g} )}^{2}}{{( {qR}_{g} )}^{4}} \qquad ( EQUATION 2 )$$

where R_{g} is the radius gyration of the peptoid chains.

Assuming that the lengths of the peptoid sheets are much greater than their lateral dimension (i.e., c>>a, b), the form factor Pₛₕₑₑₜ(q) is given by

$${P}_{sheet} ( q ) = {F}_{c} ( q ) \frac{1}{2 π} {\int }_{0}^{2 π} {{A}_{sheet} ( q , α )}^{2} d α \qquad ( EQUATION 3 )$$

where the amplitude is given by

$$\begin{matrix}{A}_{sheet} ( q , α ) = \frac{sin ( qb cos ( α ) / 2 )}{qb cos ( α ) / 2} \cdot \frac{sin ( qa sin ( α ) / 2 )}{qa sin ( α ) / 2 )} \\ and & ( EQUATION 4 ) \\ {F}_{c} ( q ) = 2 Si ( qc ) / ( qc ) - 4 {sin}^{2} ( qc / 2 ) / {( qc )}^{2} \\ where \\ Si ( x ) = {\int }_{0}^{x} {t}^{- 1} sin t dt . & ( EQUATION 5 )\end{matrix}$$

b. Self Assembled Peptides in Solution: Peptide Cylindrical Bundle Model

Each peptoid involved in the bundle is approximated as a simple solid cylinder given by:

$${P ( q )}_{cyl} = {\int }_{0}^{π / 2} {\vert {A ( q , α )}_{cyl} \vert}^{2} sin α d α ; \\ {A ( q , α )}_{cyl} = \frac{2 {J}_{1} ( qR sin α )}{qR sin α} \frac{sin ( qL cos α / 2 )}{qL cos α / 2} \qquad ( EQUATION 6 )$$

where L is the total length of the cylinder, R is the radius and a is the angle between the momentum transfer vector q and the cylinder axis parallel to L. J₁ is the first order Bessel function. The form factor describing the scattering for a bundle consisting of parallel cylinders can be calculated using the expression given by Oster and Riley:

$${P ( q )}_{bund} = \frac{{P ( q )}_{cyl}}{{N}_{cyl}^{2}} {\sum }_{i = 1}^{{N}_{cyl}} {\sum }_{j = 1}^{{N}_{cyl}} {J}_{0} ( {qd}_{ij} ) \qquad ( EQUATION 7 )$$

where J₀ is the zeroth order Bessel function and dᵢⱼ is the distance between the centers of the different cylinders. The above expression gives a generic expression that may, in principle, be evaluated for an arbitrary collection and number of cylinders. Here, a tetrameric bundle of cylinders has been utilized, where it is assumed that the bundles are arranged in a square with the center of each cylinder located in each corner. All inter-cylinder distances are then d=2fR, except for the diagonal distance which is d=√8fR, where f is a swelling factor that regulates the distance between the cylinders. EQUATION 7 can be rewritten in terms of a form factor, P(q)_{cyl} and the structure factor S(q)_{bund0}⁽ⁱ⁾ as follows:

$${P ( q )}_{bund}^{( i )} = {P ( q )}_{cyl} {S ( q )}_{bund 0}^{( i )} ; \\ {S ( q )}_{bund 0}^{( i )} = \frac{1}{16} ( 4 + 8 {J}_{0} ( 2 qRf ) + 4 {J}_{0} ( \sqrt{8} qRf ) ) \qquad ( EQUATION 8 )$$

The interaction between peptoid bundles seen at some of the highest concentrations may be modeled using an expression from the polymer reference interaction site model (PRISM) given by the structure factor [44]:

$${S}_{PRISM} ( q ) = \frac{1}{1 + vc ( q ) {P}_{cyl} ( q )} \qquad ( EQUATION 9 )$$

where v is a measure of the excluded volume and c(q) is the form factor of an infinitely thin rod [45]:

$${P}_{rod} ( q ) = 2 Si \frac{qL}{qL} - \frac{4 {sin}^{2} ( qL / 2 )}{{q}^{2} {L}^{2}} \qquad ( EQUATION 10 )$$

The total expression for the intensity is given by:

$$I = \frac{ϕ}{{V}_{p}} \cdot {V}_{p}^{2} \cdot Δ {ρ}^{2} {P}_{bound} ( q ) \cdot {S}_{PRISM} ( q ) \cdot {f}_{agg} + ( 1 - {f}_{agg} ) \cdot {P}_{chain} ( q ) \qquad ( EQUATION 11 )$$

where f_{agg} is the fraction of peptoid chains aggregated in bundles, allowing for calculation of a CAC from ϕ_{CAC}=ϕ−(f_{agg}*ϕ).

### C. Nanostructure of Peptoids in Solution-SAXS Results

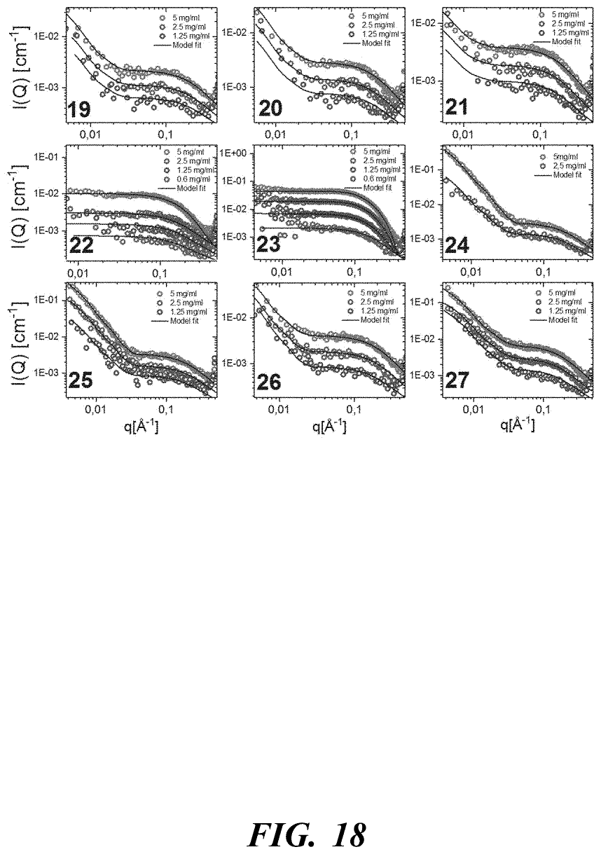

FIG. 18 depicts full SAXS results for all 10-mers measured at ESRF in the indicated concentrations together with model fits (compounds 19-21 and 24-27 were fitted using the Random polymer-like chains with fiber-like clusters model (EQUATION 1), while compounds 22 and 23 were fitted using the bundle model (EQUATION 6)). The full fit parameters are set forth in TABLES 2-3 below.

TABLE 2
Full fit parameters for the 10-mers.

| Compound | CAC | Rg* | Fraction of sheets | Dimensions: | Model |
|---|---|---|---|---|---|
| 19 | >5 mg/mL | 6.8Å | <0.001% sheets (consistent | Sheet | Gaussian |
| | | | for concentration range 5- | dimensions: | chains (with |
| | | | 1.25 mg/mL | 560Å × 150Å × | sheets) |
| | | | | > 600Å | |

Fully halogenated:

| | | | | | |
|---|---|---|---|---|---|
| 20 | >5 mg/mL | 7.5Å | <0.001% sheets (consistent | Sheet | Gaussian |
| | | | for concentration range 5- | dimensions: | chains (with |
| | | | 1.25 mg/mL) | 550Å × 145Å × | sheets) |
| | | | | > 600Å | |
| 21 | >5 mg/mL | 8.7Å | <0.0005% sheets (consistent | Sheet | Gaussian |
| | | | for concentration range 5- | dimensions: | chains (with |
| | | | 1.25 mg/mL) | 550Å × 190Å × | sheets) |
| | | | | > 600Å | |
| 22 | 2.3 mg/mL | 9.4Å | — | | Bundles |
| 23 | 0.4 mg/mL | 9Å | — | | Bundles |

Half halogenated:

| | | | | | |
|---|---|---|---|---|---|
| 24 | >5 mg/mL | 7.7Å | 0.006% sheets at 5 mg/mL and | Sheet | Gaussian |
| | | | 0.002% for 2.5 mg/mL | dimensions: | chains (with |
| | | | | 550Å × 150Å × | sheets |
| | | | | > 600Å | |
| 25 | >5 mg/mL | 7.7Å | 0.004% sheets at 5 mg/mL, | Sheet | Gaussian |
| | | | 0.003% for 2.5 mg/mL and | dimensions: | chains (with |
| | | | 0.001% for 1.25 mg/mL | 580Å × 150Å × | sheets |
| | | | | > 600Å | |
| 26 | >5 mg/mL | 7.8Å | 0.0005% sheets at 5 mg/mL, | Sheet | Gaussian |
| | | | 0.0006% for 2.5 mg/mL and | dimensions: | chains (with |
| | | | 0.001% for 1.25 mg/mL | 550Å × 200Å × | sheets |
| | | | | > 600Å | |
| 27 | >5 mg/mL | 9.1Å | <0.002% sheets (consistent | Sheet | Gaussian |
| | | | for concentration range 5- | dimensions: | chains (with |
| | | | 1.25 mg/mL) | 550Å × 160Å × | sheets |
| | | | | > 600Å | |

*The Rg of the fraction free Gaussian chains.

TABLE 3
Full fit parameters for the fully Iodinated peptoids with increasing legth.

| | | CAC | Rg* | R | L | | | |
|---|---|---|---|---|---|---|---|---|
| Compound | n-mers | (mg/mL) | (Å) | (Å) | (Å) | v | fa | Model |
| 5 | 6 | 2.8 | 8 | 3.7 | 24 | 0.2 | 7.1 | Bundles |
| 14 | 8 | 1.4 | 8 | 4.5 | 26 | 0.3-0.6 | 6.6 | Bundles |
| 23 | 10 | 0.5 | 9 | 4.2 | 34 | 0.3-1 | 6.7 | Bundles |
| 32 | 12 | 0.4 | 13 | 4 | 56 | 1-1.1 | 5.7 | Bundles |

*The Rg of the fraction free Gaussian chains.

### Example 1

This example illustrates the effects of halogen substitution on peptoid self-assembly in solution.

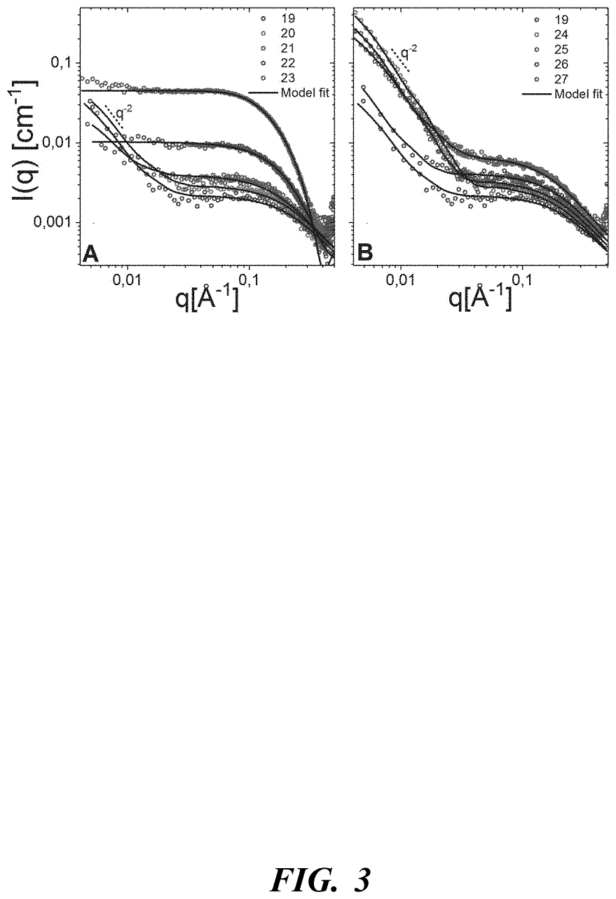

To further understand the impacts of variation in length, size of halogen groups and degree of substitution, the nanostructures of these compounds were studied in detail using Small Angle X-ray Scattering (SAXS). The results are depicted in FIG. 3.

SAXS allows for the determination of whether these peptoids self-assemble into nanostructures or exist instead as single molecules in aqueous solution. [33-36]Furthermore, through detailed theoretical modelling, the technique allows for an accurate estimation of molecular weight and shape, as well as an estimation of the overall physical structures of the peptoid assemblies. The results revealed that the observed structures depend on the length and hydrophobicity of the various peptoids; and self-assembly into defined nanostructures was observed for a few of them. Scattering intensity is plotted as a function of the modulus of the scattering vector, q=4π sin(θ/2)/λ, where λ is the wavelength of the X-rays and θ is the scattering angle (see FIG. 3). It should be noted that 1/q has the dimension of length and the quantity represents a sort of ‘measuring stick’; at low q, large structures are probed, while at high q, SAXS is sensitive to more local structures.

For the 10-mers (compounds 19-27), the scattering curve for the fully brominated and fully iodinated peptoids (compounds 22 and 23, respectively) exhibited significantly higher intensity and a different shape as compared to the rest of the 10-mers (see FIGS. 3A and 3B). The latter exhibited a typical polymer-like scattering pattern for random (Gaussian) chains, although an upturn at low q revealed a small fraction of larger structures or aggregates. The upturn follows a power law of ˜q⁻² indicating plate-like fibers, and no larger aggregates that would typically follow the power law of ˜q⁴. Fit analysis of these data using a model with a combination of free chains and rectangular fibers yielded a radius of gyration (Rg) of the free chains of 7-9 Å and a small mole fraction of only 0.001-0.0005% fiber-like sheets (see supporting information TABLE 2 for a full list of fit parameters).

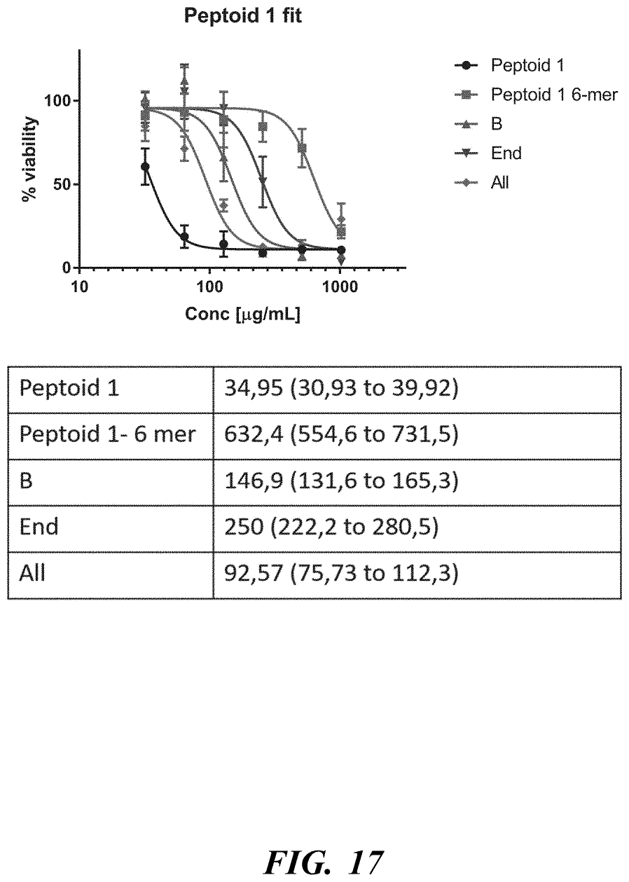

The scattering for the fully brominated and fully iodinated peptoids (compounds 22 and 23, respectively) did not exhibit the same upturn at low q and overall shape at intermediate and high q could therefore not be explained with the fit model described above. Instead, the scattering intensity exhibited a flatter q-dependence at low q indicating discrete, smaller nanostructures. The data from both compounds could be analyzed using a bundle model where cylinders representing folded/helical units are assembled into trimeric/tetrameric bundles. [34] The scattering of a concentration range from 5-0.6 mg/mL of both systems was analyzed simultaneously and the obtained fit parameters are listed in TABLE 2. The fit analysis also revealed the critical aggregation concentration (CAC) for the self-assembled structures and indicated a CAC value of 2.3 mg/mL and 0.5 mg/mL for the fully brominated and fully iodinated peptoids (compounds 22 and 23, respectively). (see FIG. 17). The low CAC value of 23 might provide an explanation of the observed loss of antimicrobial activity for the fully iodinated 10- and 12-mer (see TABLE 1) as more peptoids are “bound up” in bundles and are less available in (the presumably) active form as monomers.

In the case of the fully brominated compound 22, the CAC was found to be higher than the MIC value, and therefore does not affect the activity as there is still a significant fraction of free peptoid chains which might interact with the bacteria.

For the fully iodinated compound 23, the CAC was found to be much lower, most likely resulting in a drastically reduced availability of free monomeric peptoids, which may explain the reduction in the antimicrobial activity of this compound. As discussed in the introduction, similar trends were seen by Chu-Kung A. and co-workers. They concluded that a CAC close to the MIC of free chains in their peptide system resulted in a lack of antimicrobial activity in the same way as we observed for compound 23. [29]

### Example 2

This example illustrates the effect hydrophobicity has on the self-assembly properties of peptoids.

To further determine whether the low CAC values resulting from the 10-mer (compound 23) provide an explanation of the observed MIC data, the 6-, 8- and 12-mers of the fully iodinated peptoids (compounds 5, 14, 23, 32, respectively) were investigated. The results are shown in FIG. 3 (see TABLE 3 for fit parameters). From the fit analysis, a CAC of 2.8 mg/mL was observed for the fully iodinated 6-mer compound 5, while a CAC of 1.4 mg/ml was observed for the 8-mer compound 14 and a CAC of 0.4 mg/ml was observed for the 12-mer compound 32.

These results show that, even at very short lengths, these peptoids self-assemble due to their high hydrophobicity, which is evident from the retention times in TABLE 1. However, the detected CAC is still highly correlated with the length of the peptoids. In particular, the CACs of the 6mer and 8mer are lower than the MIC values for these compounds, so no clear link between CAC and MIC could be established. This is in contrast with the fatty acid conjugated peptides studied by Chu-Kung A. and co-workers, who found such a correlation. [29] The reduction of activity seen for the 10-mer and 12-mer is therefore likely related to their increase in hydrophobicity, indicating that there is a threshold to stay within than the self-assembly properties themselves. However, further studies into the consequences of hydrophobicity and self-assembly with the activity and toxicity for peptoids are needed to fully explain the observed trends.

### Example 3

This example illustrates the effect that halogenation has on the antimicrobial potency of peptoids.

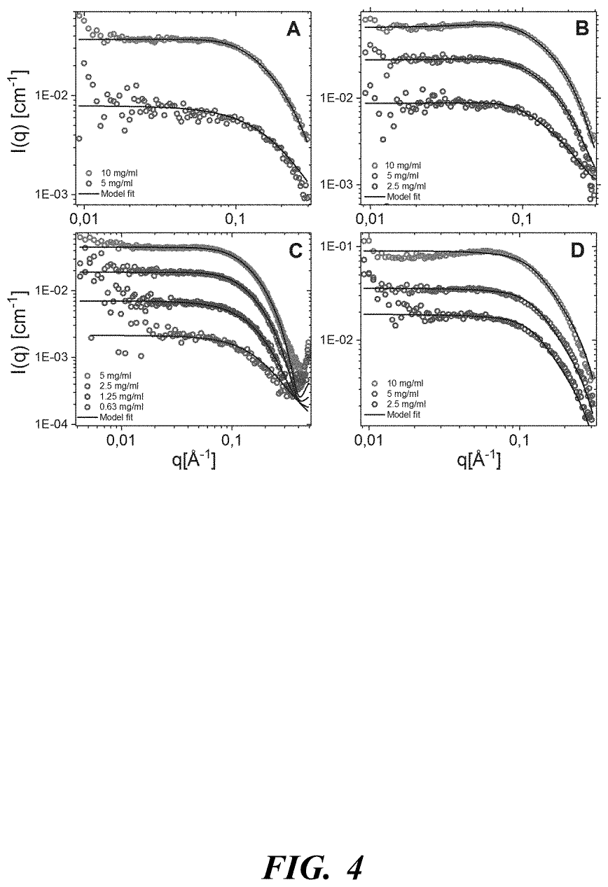

In order to investigate whether halogen substitution can be used as a tool to improve the antimicrobial potency of a known peptoid, two small libraries of chlorinated and brominated analogues of a well-studied peptoid (Peptoid 1) were synthesized (these halogenated analogs demonstrated overall higher potency compared to the fluorinated ones, and the iodine analogs raised concerns about aggregation and loss of activity). Both halogen atoms were introduced via Nspe units in position 4 on the phenyl rings. The level of substitution varied between full substitution (all phenyl rings bear a halogen atom) and “half” substitution (every other phenyl ring bears a halogen atom). This strategy yielded six chloro- and six bromo-modified versions of Peptoid 1, which are depicted in FIG. 4.

The library of 12 compounds (compounds 37-48) was tested against the same panel of bacterial strains as the first generation of peptoids. However, none of the modifications led to increased potency, while mostly caused a loss of activity (see TABLE 4 below). Judging from long HPLC high retention times, this may be explained by the fact that the critical hydrophobicity level was reached, which caused aggregation and loss of activity similar to that observed with compounds 23 and 32 (notably, the highest activity was observed for the compounds with lower retention times). Interestingly, compounds 38 and 40-42 have the same number of chlorine atoms, but their hydrophobicities slightly differ. For the brominated derivatives (compounds 44 and 46-48), this effect is was even more pronounced. It shows that even distribution of halogen atoms across the peptoid chain leads to higher hydrophobicity compared to allocating positioning chlorine or bromine atoms at the terminal ends or at the middle of the peptoid sequence.

TABLE 4
MICs (μg/mL) of the chlorinated and brominated analogues of peptoid 1.

| cmpd | RP-HPLC RT | SA^{a} | MRSA^{b} | MRSE^{c} | MRSE^{d} | EC^{e} | PA^{f} |
|---|---|---|---|---|---|---|---|
| Peptoid 1 | 15.46 | 8 | 8 | 4 | 8 | 8 | 16 |
| 37 | 18.25 | >64 | 64 | 64 | >64 | >64 | >64 |
| 38 | 16.80 | 32 | 16 | 32 | 16 | 64 | >64 |
| 39 | 16.93 | 16 | 16 | 16 | 16 | 64 | >64 |
| 40 | 16.09 | 8 | 8 | 8 | 8 | >64 | 16 |
| 41 | 16.20 | 8 | 8 | 8 | 8 | 64 | 16 |
| 42 | 16.86 | 16 | 16 | 16 | 16-32 | 64 | >64 |
| 43 | 18.88 | >64 | >64 | >64 | >64 | >64 | >64 |
| 44 | 17.03 | 64 | 32 | 32 | 32 | 64 | >64 |
| 45 | 17.21 | 32 | 32 | 32 | 64 | >64 | >64 |
| 46 | 16.23 | 16 | 8 | 8 | 8 | >64 | 16 |
| 47 | 15.68 | 8 | 8 | 4 | 8 | 64 | 16 |
| 48 | 17.14 | 64 | 32 | 32 | 32 | 64 | >64 |

^{a}S. aureus ATCC 25923;
^{b}methicillin-resistant S. aureus USA 300;
^{c}methicillin-restant S. epidermidis ET-024;
^{d}methicillin-restant S. epidermidis ATCC 51625;
^{e}E. coli ATCC 25922;
^{f}P. aeruginosa PA01 H103;
RP-HPLC RT-Reversed-Phase HPLC retention time.

### Example 3

This example illustrates the effect of halogenation on the antimicrobial activity of non-active short analogs (the Pep1-6mer) of Peptoid 1.

As seen from the increased values of retention times, the hydrophobicity ceiling has been hit during the introduction of halogens into the sequence of peptoid Peptoid 1 (see TABLE 4). Hence, the effect of shortening the length of Peptoid 1 from twelve to six residues (cutting its length in half) was investigated. To this end, a small library of four analogues was synthesized with different levels of halogenation (see FIG. 4). As seen from TABLES 3 and 4, introduction of fluorine did not result in the desired increase of activity, while bromination had a more pronounced effect on the hydrophobicity than chlorination and similar to one displayed by the iodine-containing peptoids. Hence, three brominated short peptoids analogs of Peptoid 1 (compounds 49-51) and one non-halogenated peptoid Pep1-6mer were synthesized and tested against the same bacterial strains as the previous peptoids. The resulting data is shown in TABLE 5 below, where the new data set is compared with the original peptoid (Peptoid 1) data.

TABLE 5
MICs (μg/mL) and IC50 (μg/mL) towards HaCaT cell line of short brominated
peptoid Peptoid 1 analogues.

| cmpd | RT | SA^{a} | MRSA^{b} | MRSE^{c} | MRSE^{d} | EC^{e} | PA^{f} | IC₅₀ (μg/mL) |
|---|---|---|---|---|---|---|---|---|
| Peptoid 1 | 3.68 | 8 | 8 | 4 | 8 | 8 | 16 | 35.0 (30.9 to 39.9) |
| Pep1-6ₘₑᵣ | 3.08 | 256 | 256 | 64 | 64 | 1024 | 256 | 632.4 (554.6 to |
| | | | | | | | | 731.5) |
| 49 | 3.59 | 64 | 32 | 16 | 8 | >64 | 64 | 250 (222.2 to |
| | | | | | | | | 280.5) |
| 50 | 3.84 | 8 | 8 | 4 | 2 | 32 | 16 | 146.9 (131.6 to |
| | | | | | | | | 165.3) |
| 51 | 4.02 | 8 | 8 | 4 | 8 | 16 | 16 | 92.6 (75.7 to 112.3) |

^{a}S. aureus ATCC 25923;
^{b}methicillin-resistant S. aureus USA 300;
^{c}methicillin-restant S. epidermidis ET-024;
^{d}methicillin-restant S. epidermidis ATCC 51625;
^{e}E. coli ATCC 25922;
^{f}P. aeruginosa PA01 H103;
RT-retention time

Compound 49 has only one terminal bromine and compound 50 has two bromine atoms. In compound 51, all four phenyl rings are substituted with a bromine atom. The addition of just two bromine atoms was enough to improve the activity of a non-active short analogue, the Pep1-6mer, 16-32-fold against both S. aureus and S. epidermidis. Incorporation of two additional bromine substituents (yielding four in total) failed to significantly affect the activity. The addition of one terminal halogen atom, on the other hand, was already enough to impart a several-fold increase in antimicrobial activity. In contrast to the previous data (where introduction of halogens did not improve the activity against Gram-negative bacteria), in this case, the addition of two bromines imparted sufficient hydrophobicity in order to obtain an MIC against P. aeruginosa that is close to that of the original peptoid (Peptoid 1), and reached a 32-fold increase of activity against E. coli compared to the Pep1-6mer.

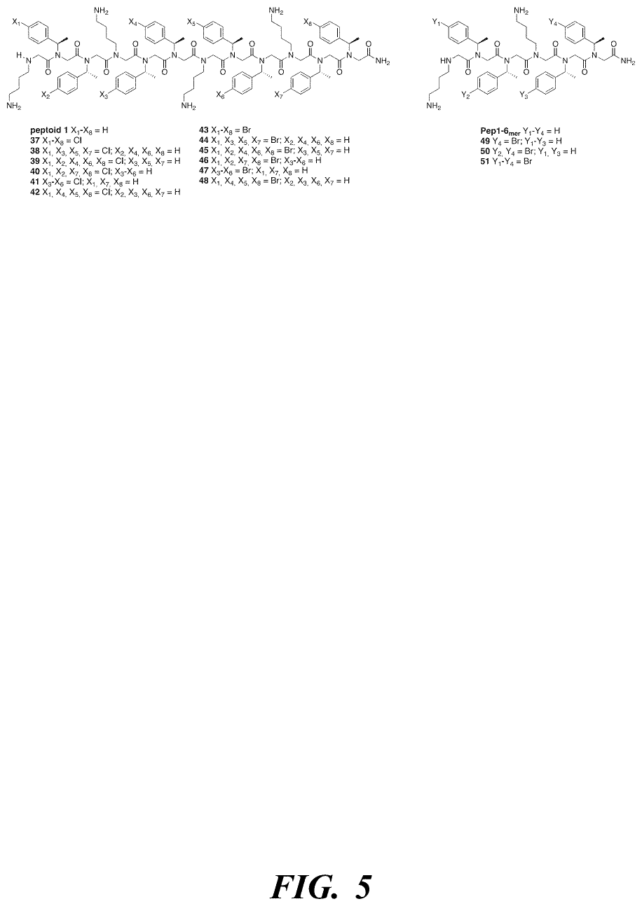

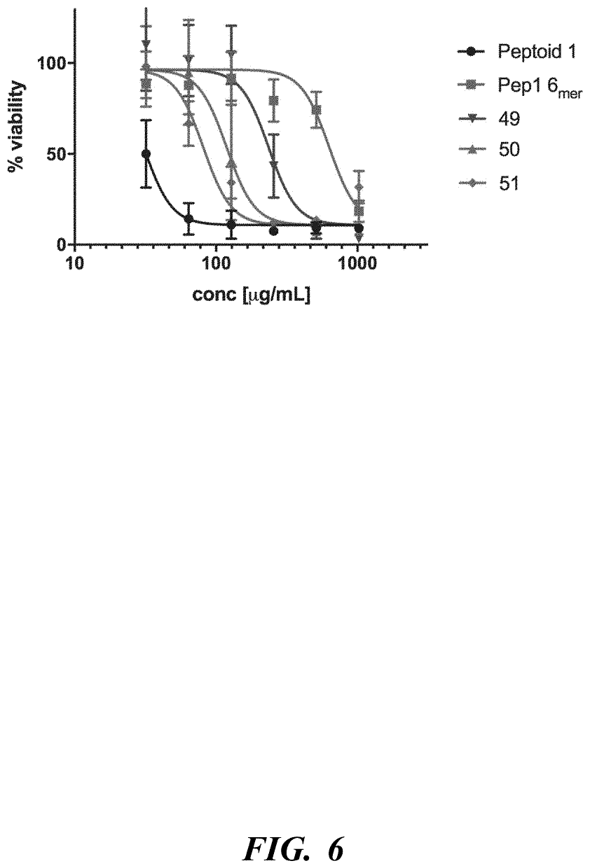

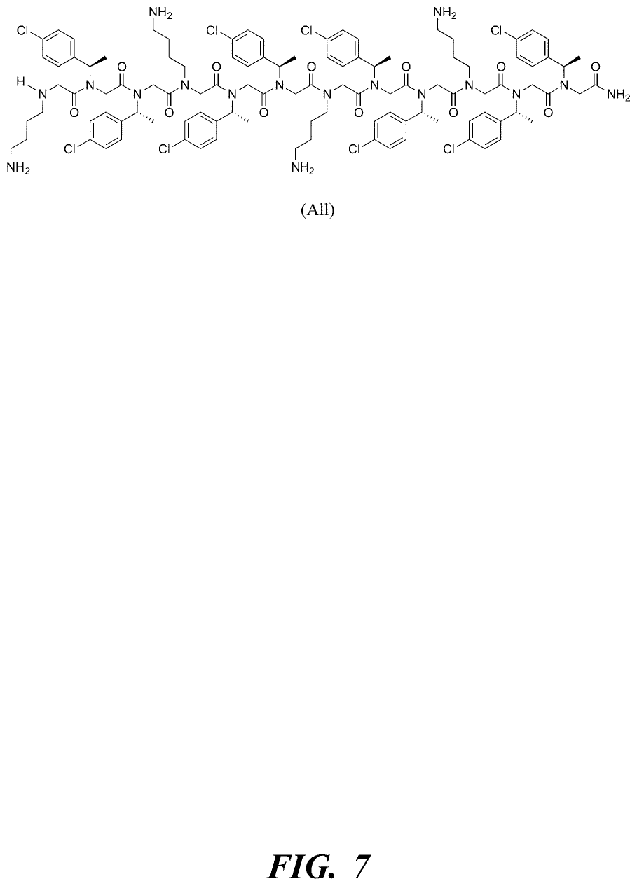

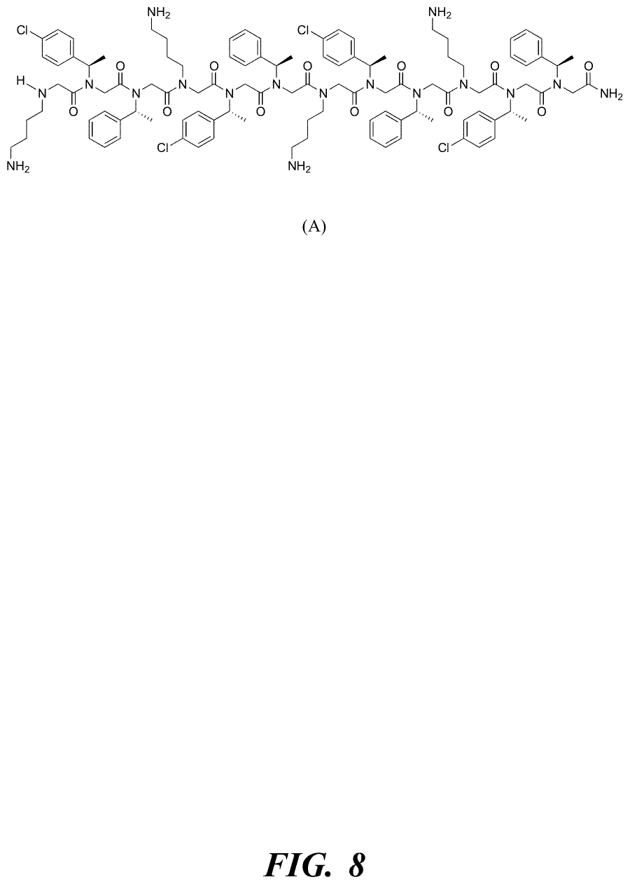

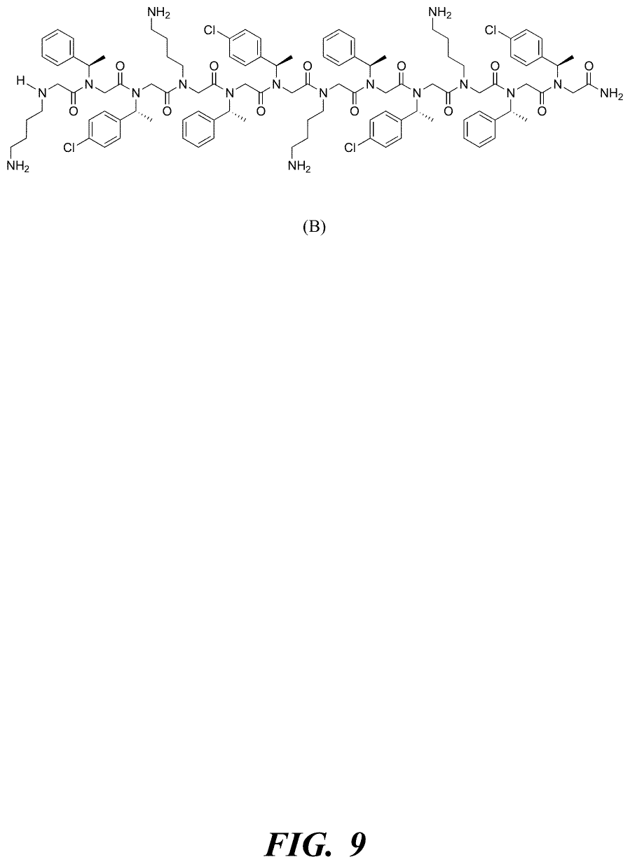

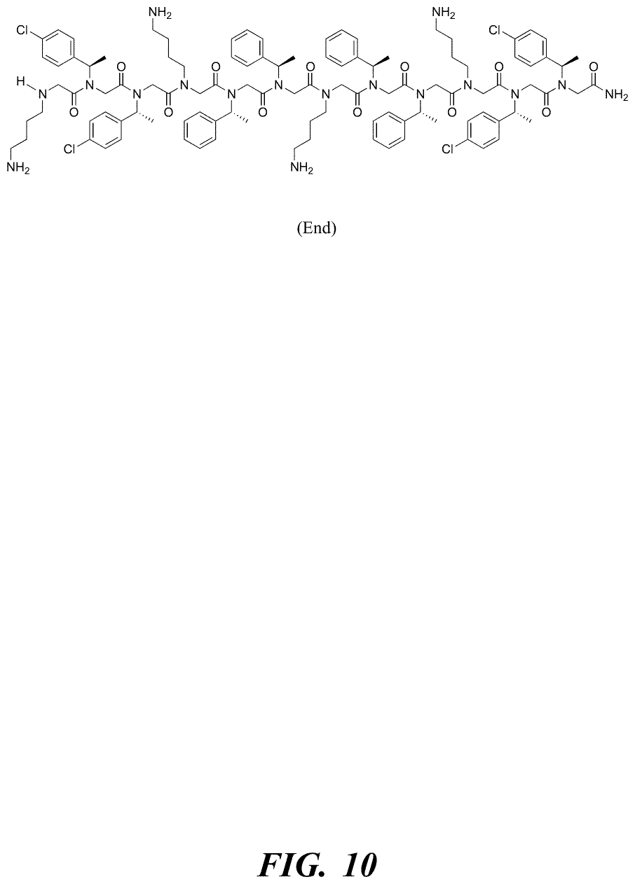

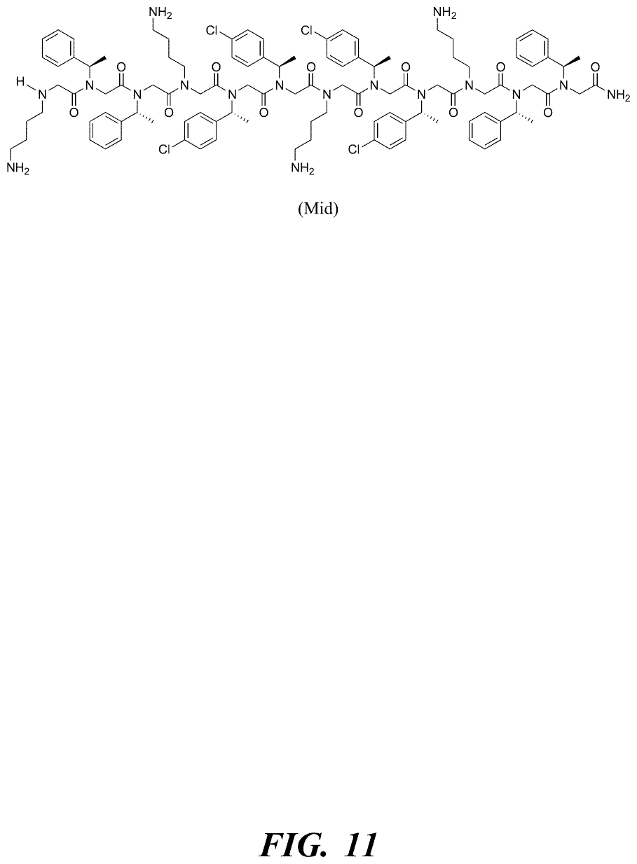

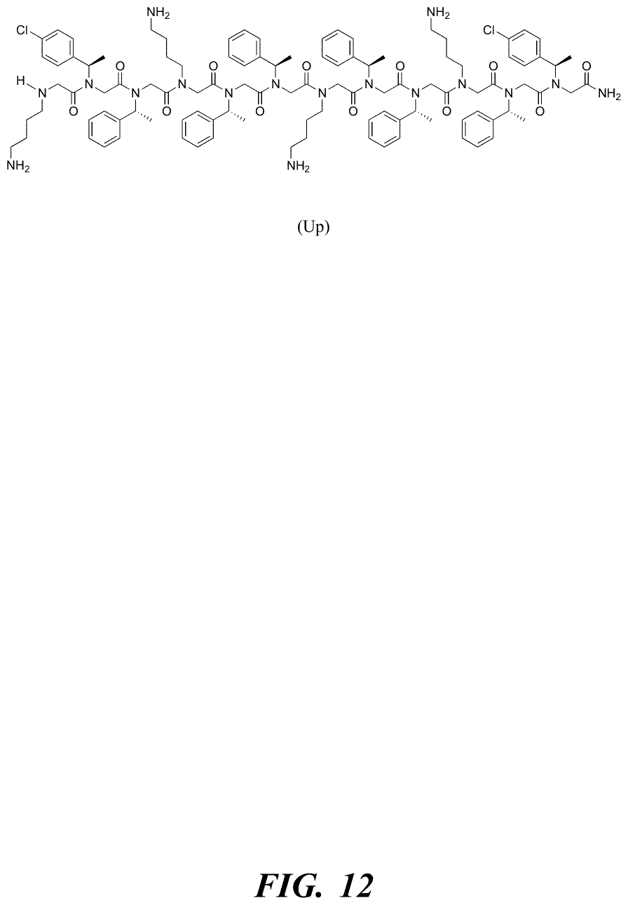

Since the incorporation of halogens was found to improve the activity of inactive peptoids, the cytotoxicity profiles of the resulting compounds were investigated. Peptoid 1, Pep1-6mer and the three brominated analogues were tested towards a HaCaT cell line for 1 hour. The results were obtained using MTS/PMS assay (see FIG. 5, TABLE 3).

A clear trend between the number of halogen atoms and corresponding increased cytotoxicity was observed. However, while compound 50 demonstrated the same antimicrobial activity profile as Peptoid 1, it was less cytotoxic, with an IC50 of 146.9 μg/mL versus only 35.0 μg/mL for the latter. Initial results indicate reduced cytotoxicity of the brominated analogues when compared to Peptoid 1, though compounds 50 and 51 displayed similar activity and hydrophobicity profiles.

### V. Conclusions

The foregoing examples demonstrate the effect of fluorine, chlorine, bromine and iodine substitution on the antimicrobial activity of peptoids. First, using an inactive model (NLys-Npm)ₙ peptoid scaffold, it was shown that the incorporation of chlorine or bromine may provide an improvement of antimicrobial activity against Gram-positive bacteria, while fluorination did not display any pronounced effect. Introduction of iodine in 6- and 8-mer analogues dramatically increased the activity, but led to loss of activity due to aggregation in 10- and 12-mers. Attempts to improve the antibacterial potency of Peptoid 1 by incorporating chlorine or bromine atoms via Nspe units led to overall loss of activity. Without wishing to be bound by theory, this suggests that hydrophobicity limits have been reached. However, bromination of a shorter inactive 6-mer analogue of Peptoid 1 resulted in the same activity as the 12-mer Peptoid 1 against some bacteria, while noticeably improving its cytotoxicity profile.

The foregoing demonstrates that halogenation (and in particular, bromination) may be used to readily modify and alter the physicochemical and antibacterial properties of peptoids, but the effect strongly depends on the choice of the halogen. In addition, the effect is sequence- and length-specific, and inclusion of halogens may also lower antimicrobial activity.

### VI. Miscellaneous

The compositions described herein may be halogenated in various ways. For example, these compounds may include any number of halogen substitutions with the same or different halogens. In particular, these compounds may include one or more fluoro-, chloro-, bromo- or iodo-substitutions, and may include substitution with two or more distinct halogens. However, the use of one or two bromo- or chloro-substitutions is preferred in many applications. Moreover, while the peptoids described herein may be halogenated at various locations, para halogenation on peptoids containing aryl rings is especially preferred in many applications, although ortho- and meta-substitution, or even perhalogentation, may be useful in some applications.

The halogenated compositions described herein may also be alkylated, and preferably have terminal alkylation. Here, alkylation (and especially terminal alkylation) with a C₁₀ or C₁₃ tail is especially preferred. It has been found that such terminal alkylation can dramatically enhance the antibacterial activity of a peptoid, and in some cases, may cause a peptoid which otherwise has low antibacterial activity to have significant antibacterial activity.

The use of poly-N-substituted glycine compounds in the compositions and methodologies described herein is preferred. Preferably, these poly-N-substituted glycine compounds are poly-N-substituted glycines having the formula

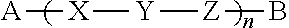

wherein

A is a terminal N-alkyl substituted glycine residue,

n is an integer,

B is selected from the group consisting of NH₂, one and two N-substituted glycine residues, and wherein said one and two N-substituted glycine residues have N-substituents which are independently selected from natural α-amino acid side chain moieties, isomers and carbon homologs thereof, and

X, Y and Z are independently selected from the group consisting of N-substituted glycine residues, wherein said N-substituents are independently selected from the group consisting of natural α-amino acid side chain moieties, isomers and carbon homologs thereof, and proline residues, and wherein at least one of A, B, X, Y and Z contains a halogen-bearing moiety. The alkyl substituent is preferably selected from about C₄ to about C₂₀ linear, branched and cyclic alkyl moieties, and n preferably has a value within the range of 1-3. Preferably, at least one of said X, Y and Z residues is N_{Lys} and at least one said N-substituent is chiral. It is also preferred that at least one of Y and Z are proline residues. It is further preferred that A is a terminal N-alkyl substituted glycine residue, wherein the alkyl substituent is selected from the group consisting of C₆ to about C₁₈ linear alkyl moieties, wherein B is NH₂, and wherein n is 1 or 2. In some embodiments, A is a terminal N-alkyl substituted glycine residue, wherein the alkyl substituent selected from about C₆ to about C₁₈ linear alkyl moieties, wherein B is an N_{Lys} residue, and wherein n is 1. In some embodiments, the compound may be a hexamer or a dodecamer.

In the foregoing poly-N-substituted glycines, the halogen-bearing moiety may be a halogen-substituted aryl moiety such as, for example, a chloro-substituted aryl moiety, a bromo-substituted aryl moiety or an iodo-substituted aryl moiety. In some embodiments, each mer may contain a halogen-substituted aryl moiety, while in other embodiments, some of the mers may contain a halogen-substituted aryl moiety, and some of the mers in the hexamer contain a halogen-free aryl moiety. In still other embodiments, exactly one of the mers contains a halogen-substituted aryl moiety. In some embodiments of the foregoing poly-N-substituted glycines, at least two of A, B, X, Y and Z contain a halogen-bearing moiety, while in other embodiments, all of A, B, X, Y and Z contain a halogen-bearing moiety. While the foregoing poly-N-substituted glycines may contain halogen substitution including any halogen, substitution with chlorine, bromine and/or iodine is preferred, and parahalogenation on aryl moieties on these poly-N-substituted glycines is especially preferred.

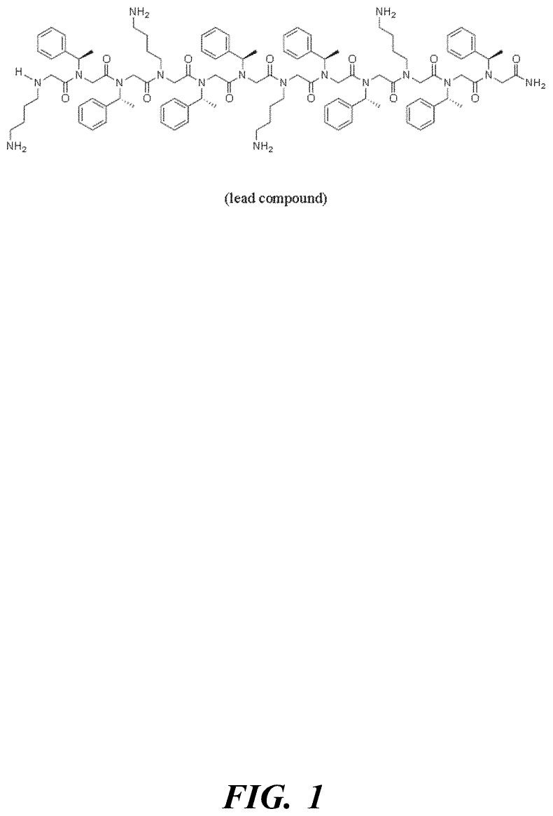

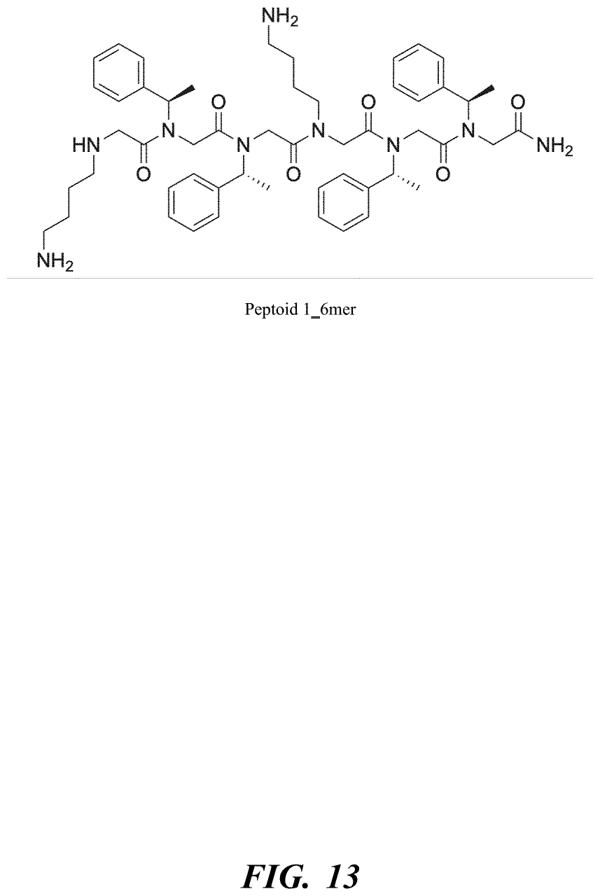

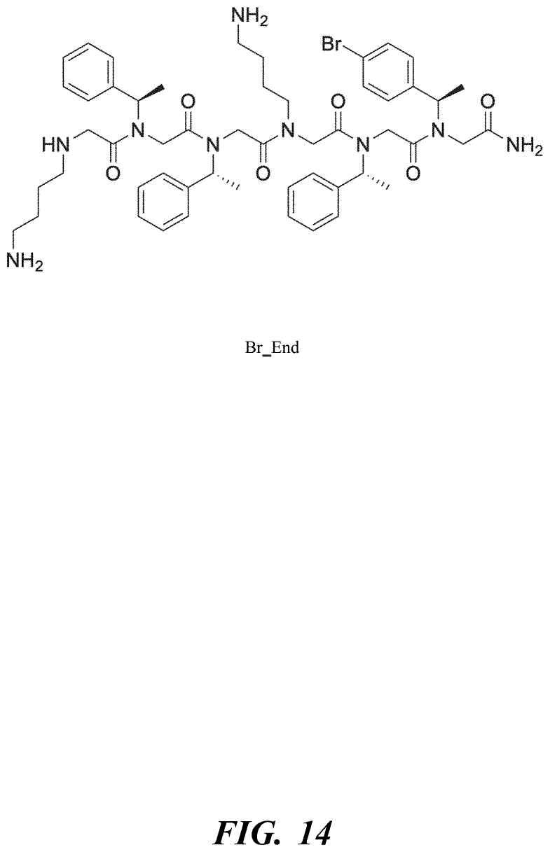

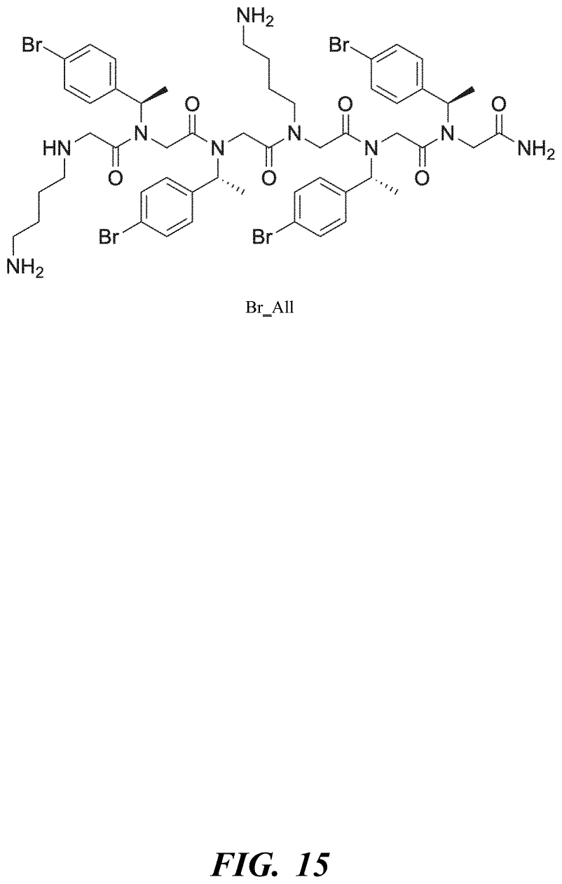

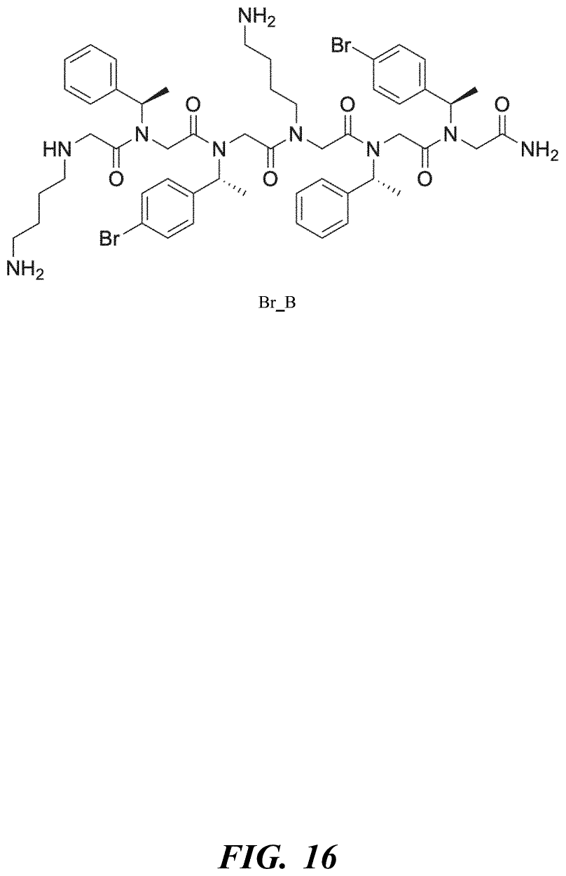

In some embodiments, the poly-N-substituted glycine may be a compound derived from a material selected from the group consisting of the compound of FIG. 1 and the compound of FIG. 13 by substituting at least one hydrogen atom in at least one aryl moiety thereof with at least one halogen atom, and more preferably by substituting at least one para-hydrogen atom in at least one aryl moiety thereof with at least one halogen atom. In some embodiments, the compound is derived from a material selected from the group consisting of the compound of FIG. 1 and the compound of FIG. 13 by substituting one hydrogen atom in each aryl moiety thereof with at least one halogen atom. In the foregoing embodiments, the at least one halogen atom is preferably selected from the group consisting of chlorine, bromine and iodine. In some embodiments, the at least one halogen atom may include at least first and second distinct halogens selected from the group consisting of chlorine, bromine and iodine. Especially preferred embodiments of these poly-N-substituted glycines include the following compounds (or pharmaceutically acceptable salts thereof):

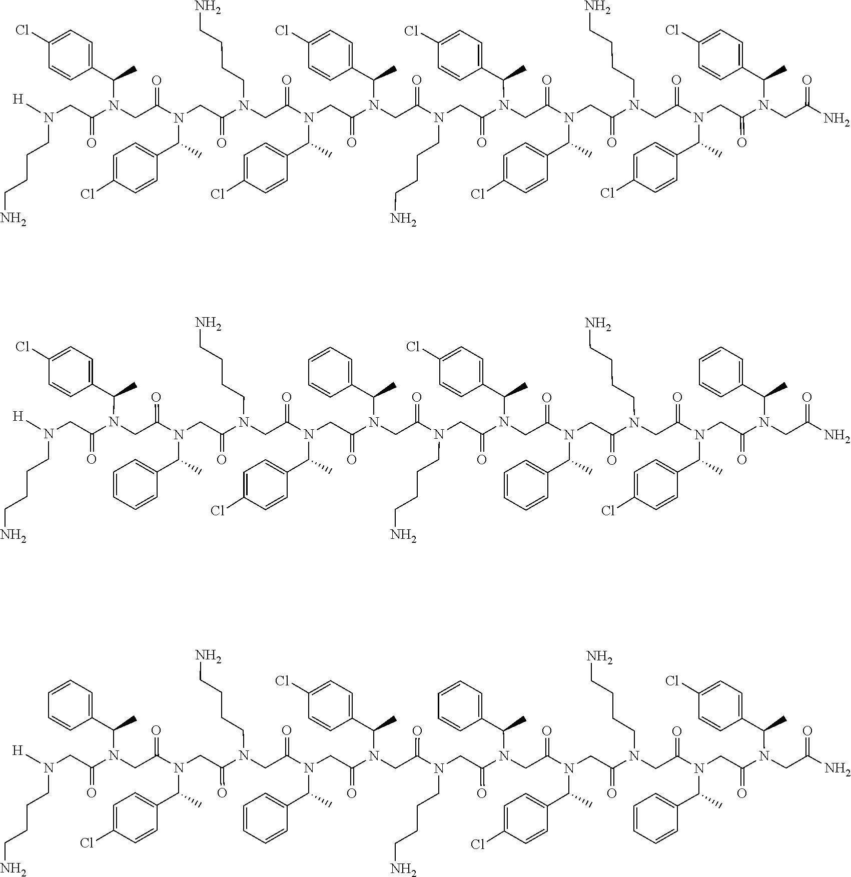

Some embodiments of the compositions and methodologies disclosed herein may utilize a poly-N-substituted glycine compound containing at least one halogen selected from the group consisting of chlorine, bromine and iodine. The poly-N-substituted glycine compound contains at least one halogen, preferably at least two halogens, and in some cases at least four halogens selected from the group consisting of chlorine, bromine and iodine. For example, the poly-N-substituted glycine compound may contain at least two bromine atoms or at least two chlorine atoms, or may contain at least four bromine atoms or at least four chlorine atoms. Preferably, the poly-N-substituted glycine compound contains at least one parahalogenated aryl group, and more preferably at least two parahalogenated aryl groups, and in some cases contains at least four parahalogenated aryl groups. In especially preferred embodiments, the poly-N-substituted glycine compound contains a carboxamide terminus group. Such a compound may be fabricated via a rink amide resin.

The peptoids described herein may be incorporated into various pharmaceutical compositions which may be utilized for various purposes, including as antibacterial, antifungal, and also possibly antiviral and antiparasitic compositions. The pharmaceutical compositions utilized in the systems and methodologies disclosed herein may utilize one or more active ingredients which may be dissolved, suspended or disposed in various media. Such media may include, for example, various liquid, solid or multistate media such as, for example, emulsions, gels or creams. Such media may include liquid media, which may be hydrophobic or may comprise one or more triglycerides or oils. Such media may include, but is not limited to, vegetable oils, fish oils, animal fats, hydrogenated vegetable oils, partially hydrogenated vegetable oils, synthetic triglycerides, modified triglycerides, fractionated triglycerides, and mixtures thereof. Triglycerides used in these pharmaceutical compositions may include those selected from the group consisting of almond oil; babassu oil; borage oil; blackcurrant seed oil; black seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; and mixtures thereof. The use of coconut oil is especially preferred.

Various fatty acids may be utilized in the pharmaceutical compositions disclosed herein. These include, without limitation, both long and short chain fatty acids. Examples of such fatty acids include, but are not limited to, docosahexaenoic acid, caprylic acid, capric acid, lauric acid, butyric acid, and pharmaceutically acceptable salts thereof.

The pharmaceutical compositions disclosed herein may be applied in various manners. Thus, for example, these compositions may be applied as oral, transdermal, transmucosal, intravenous or injected treatments, or via cell-based drug delivery systems. Moreover, these compositions may be applied in a single dose, multi-dose or controlled release fashion.

The pharmaceutical compositions disclosed herein may be manufactured as tablets, liquids, gels, foams, ointments or powders. In some embodiments, these compositions may be applied as microparticles or nanoparticles, via aerosols or sprays, or as dispersed micelles which contain self-assembled peptoids in the interiors of the micelles (which would be overall water-soluble).

Various counterions may be utilized in forming pharmaceutically acceptable salts of the materials disclosed herein. One skilled in the art will appreciate that the specific choice of counterion may be dictated by various considerations. However, the use of sodium and hydrochloride salts may be preferred in some applications. In general, self-assembling peptoids will be initially dissolved in the absence of divalent counterions such as manganese, magnesium, calcium, etc.

In some embodiments, the compositions described herein may be formulated as mixtures of different peptoids compounds. For example, in some embodiments, mixtures of two or more halogenated peptoids of the type disclosed herein may be formed. In other embodiments, mixtures of one or more of the halogenated peptoids described herein may be formed with one or more nonhalogenated peptoids including, for example, the peptoids described in U.S. Pat. No. 8,445,632 (Barron et al.), entitled Selective Poly-N-Substituted Glycine Antibiotics”, which is incorporated herein by reference in its entirety. It is also to be noted that halogenated analogs to any of the peptoids disclosed in the '632 patent may be produced in accordance with the teachings herein.

Various cyclic peptoids may be produced in accordance with the teachings herein. These include, but are not limited to, halogenated analogs of the cyclic peptoids disclosed in U.S. Pat. No. 9,938,321 (Kirshenbaum et al.), U.S. Pat. No. 9,315,548 (Kirshenbaum et al.) and U.S. Pat. No. 8,828,413 (Kirshenbaum et al.), all of which are incorporated herein by reference in their entirety. These halogen analogs may feature halogen substitution on one or more of the ring structures by one or more halogens, but preferably include bromo-substituted or chloro-substituted analogs.

The compositions and methodologies disclosed herein may be utilized in the treatment of various diseases caused by a variety of pathogens. These treatments may utilize various other pharmaceutically active or effective materials such as, for example, pulmonary lung surfactants, collectins, peptides, peptoids, peptidomimetics, aminoglycoside antibiotics, or vaccines. The pathogens treatable with these therapies may include viruses (including, but not limited to, SARS-CoV-2), bacteria (including gram-positive and gram-negative bacteria), fungi, and parasites.

Diseases of various etymologies may be treated with the compositions and methodologies disclosed herein. Examples of such diseases of a fungal etymology include, but are not limited to, aspergillosis; candidiasis; mucormycosis; histoplasmosis; blastomycosis; coccidioidomycosis; and paracoccidioidomycosis. Examples of such diseases of a bacterial etymology include, but are not limited to, brucellosis; Campylobacter infections; cat-scratch disease; chlamydial infections; cholera; Escherichia coli infections; gonorrhea; Klebsiella, Enterobacter, and Serratia infections; Legionella infections; meningococcal infections; pertussis, plague, Mycobacterium tuberculosis infections, Pseudomonas infections; Salmonella infections; shigellosis; typhoid fever; and tularemia; anthrax; diphtheria; enterococcal infections; erysipelothricosis; listeriosis; nocardiosis; pneumococcal infections; staphylococcal infections; streptococcal infections; spirochete infections such as Borrelia Burgdorferri, bejel, yaws, and pinta; leptospirosis; Lyme disease; rat-bite fever; relapsing fever; syphilis; actinomycosis; Bacteroides; botulism; clostridial infections; and tetanus. Examples of such diseases of a viral etymology include, but are not limited to, infections caused by enveloped RNA viruses such as, for example, coronavirus infections (including those caused by alpha coronaviruses and beta coronaviruses, and specifically including those caused by SARS-CoV-2), including severe acute respiratory syndrome (SARS), Middle East respiratory syndrome (MERS) and coronavirus disease 19 (COVID-19); enterovirus infections; bornavirus infections, herpesvirus infections; cytomegaloviruses such as HHV6A and HHV7, hepatitis A; hepatitis B; hepatitis C, Epstein-Barr virus, human papillomavirus (HPV); influenza; Japanese encephalitis (inflammation of the brain); measles, mumps, and rubella; polio; rabies; rotavirus; varicella; shingles (herpes zoster); and yellow fever), and HIV-1. Parasitic infections may include those involving Toxoplasma gondii and Trypanosoma cruzi.

The above description of the present invention is illustrative, and is not intended to be limiting. It will thus be appreciated that various additions, substitutions and modifications may be made to the above described embodiments without departing from the scope of the present invention. Accordingly, the scope of the present invention should be construed in reference to the appended claims. For convenience, some features of the claimed invention may be set forth separately in specific dependent or independent claims. However, it is to be understood that these features may be combined in various combinations and subcombinations without departing from the scope of the present disclosure. By way of example and not of limitation, the limitations of two or more dependent claims may be combined with each other without departing from the scope of the present disclosure.

### REFERENCES

1. Bassetti, M.; Merelli, M.; Temperoni, C.; Astilean, A., New antibiotics for bad bugs: where are we? Ann Clin Microbiol Antimicrob 2013, 12, 22.
2. Mulani, M. S.; Kamble, E. E.; Kumkar, S. N.; Tawre, M. S.; Pardesi, K. R., Emerging Strategies to Combat ESKAPE Pathogens in the Era of Antimicrobial Resistance: A Review. Front Microbiol 2019, 10, 539.
3. Chen, C. H.; Lu, T. K., Development and Challenges of Antimicrobial Peptides for Therapeutic Applications. Antibiotics (Basel) 2020, 9 (1).
4. WHO. “Critically Important Antimicrobials for Human Medicine.” 6th.
5. Dickey, S. W.; Cheung, G. Y. C.; Otto, M., Different drugs for bad bugs: antivirulence strategies in the age of antibiotic resistance. Nat Rev Drug Discov 2017, 16 (7), 457-471.
6. Friedman, N. D.; Temkin, E.; Carmeli, Y., The negative impact of antibiotic resistance. Clin Microbiol Infect 2016, 22 (5), 416-22.
7. Zasloff, M., Antimicrobial peptides of multicellular organisms. Nature 2002, 415 (6870), 389-95.
8. H. B. Koo, J. S., Antimicrobial peptides under clinical investigation. Peptide Science 2019, 111:e24122.
9. Mahlapuu, M.; Hakansson, J.; Ringstad, L.; Bjorn, C., Antimicrobial Peptides: An Emerging Category of Therapeutic Agents. Front Cell Infect Microbiol 2016, 6, 194.
10. Zuckermann R N, K. J., Kent S B H, Moos W H, Efficient method for the preparation of peptoids [oligo(N-substituted glycines)] by submonomer solid-phase synthesis. J Am Chem Soc. 1992, 114 (2).
11. Molchanova, N.; Hansen, P. R.; Franzyk, H., Advances in Development of Antimicrobial Peptidomimetics as Potential Drugs. Molecules 2017, 22 (9).
12. Neumann, C. S.; Fujimori, D. G.; Walsh, C. T., Halogenation strategies in natural product biosynthesis. Chem Biol 2008, 15 (2), 99-109.
13. Gribble, G. W., Natural organohalogens: a new frontier for medicinal agents? J Chem Educ 2004, 81 (10), 1441-1449.
14. Gimenez, D.; Andreu, C.; del Olmo, M.; Varea, T.; Diaz, D.; Asensio, G., The introduction of fluorine atoms or trifluoromethyl groups in short cationic peptides enhances their antimicrobial activity. Bioorg Med Chem 2006, 14 (20), 6971-8.
15. Meng, H.; Kumar, K., Antimicrobial activity and protease stability of peptides containing fluorinated amino acids. J Am Chem Soc 2007, 129 (50), 15615-22.
16. Paulsen, M. H.; Karlsen, E. A.; Ausbacher, D.; Anderssen, T.; Bayer, A.; Ochtrop, P.; Hedberg, C.; Haug, T.; Ericson Sollid, J. U.; Strom, M. B., An amphipathic cyclic tetrapeptide scaffold containing halogenated beta(2,2)-amino acids with activity against multiresistant bacteria. J Pept Sci 2018, 24 (10), e3117.
17. Gottler, L. M.; de la Salud Bea, R.; Shelburne, C. E.; Ramamoorthy, A.; Marsh, E. N., Using fluorous amino acids to probe the effects of changing hydrophobicity on the physical and biological properties of the beta-hairpin antimicrobial peptide protegrin-1. Biochemistry 2008, 47 (35), 9243-50.
18. Findlay, B.; Zhanel, G. G.; Schweizer, F., Investigating the antimicrobial peptide ‘window of activity’ using cationic lipopeptides with hydrocarbon and fluorinated tails. Int J Antimicrob Agents 2012, 40 (1), 36-42.
19. Molchanova, N.; Hansen, P. R.; Damborg, P.; Nielsen, H. M.; Franzyk, H., Lysine-Based alpha-Peptide/beta-Peptoid Peptidomimetics: Influence of Hydrophobicity, Fluorination, and Distribution of Cationic Charge on Antimicrobial Activity and Cytotoxicity. ChemMedChem 2017, 12 (4), 312-318.
20. Molchanova, N.; Hansen, P. R.; Damborg, P.; Franzyk, H., Fluorinated antimicrobial lysine-based peptidomimetics with activity against methicillin-resistant Staphylococcus pseudintermedius. J Pept Sci 2018, 24 (7), e3098.
21. Harris, C. M.; Kannan, R.; Kopecka, H.; Harris, T. M., The Role of the Chlorine Substituents in the Antibiotic Vancomycin—Preparation and Characterization of Monodechlorovancomycin and Didechlorovancomycin. Journal of the American Chemical Society 1985, 107 (23), 6652-6658.
22. Groll, M.; Huber, R.; Potts, B. C., Crystal structures of Salinosporamide A (NPI-0052) and B (NPI-0047) in complex with the 20S proteasome reveal important consequences of beta-lactone ring opening and a mechanism for irreversible binding. J Am Chem Soc 2006, 128 (15), 5136-41.
23. Jia, F.; Zhang, Y.; Wang, J.; Peng, J.; Zhao, P.; Zhang, L.; Yao, H.; Ni, J.; Wang, K., The effect of halogenation on the antimicrobial activity, antibiofilm activity, cytotoxicity and proteolytic stability of the antimicrobial peptide Jelleine-I. Peptides 2019, 112, 56-66.
24. Cruz, J. C.; Iorio, M.; Monciardini, P.; Simone, M.; Brunati, C.; Gaspari, E.; Maffioli, S. I.; Wellington, E.; Sosio, M.; Donadio, S., Brominated Variant of the Lantibiotic NAI-107 with Enhanced Antibacterial Potency. J Nat Prod 2015, 78 (11), 2642-7.
25. Lee, J.; Kang, D.; Choi, J.; Huang, W.; Wadman, M.; Barron, A. E.; Seo, J., Effect of side chain hydrophobicity and cationic charge on antimicrobial activity and cytotoxicity of helical peptoids. Bioorg Med Chem Lett 2018, 28 (2), 170-173.
26. Gentry, C. L.; Egleton, R. D.; Gillespie, T.; Abbruscato, T. J.; Bechowski, H. B.; Hruby, V. J.; Davis, T. P., The effect of halogenation on blood-brain barrier permeability of a novel peptide drug. Peptides 1999, 20 (10), 1229-38.
27. Haffner S. M., M. M., Influence of self-assembly on the performance of antimicrobial peptides. Current Opinion in Colloid & Interface Science 2018, 38, 56-79.
28. Tian, X.; Sun, F.; Zhou, X. R.; Luo, S. Z.; Chen, L., Role of peptide self-assembly in antimicrobial peptides. J Pept Sci 2015, 21 (7), 530-9.
29. Chu-Kung, A. F.; Nguyen, R.; Bozzelli, K. N.; Tirrell, M., Chain length dependence of antimicrobial peptide-fatty acid conjugate activity. J Colloid Interface Sci 2010, 345 (2), 160-7.
30. Xu, D.; Jiang, L.; Singh, A.; Dustin, D.; Yang, M.; Liu, L.; Lund, R.; Sellati, T. J.; Dong, H., Designed supramolecular filamentous peptides: balance of nanostructure, cytotoxicity and antimicrobial activity. Chem Commun (Camb) 2015, 51 (7), 1289-92.
31. Frederiksen, N.; Hansen, P. R.; Bjorkling, F.; Franzyk, H., Peptide/Peptoid Hybrid Oligomers: The Influence of Hydrophobicity and Relative Side-Chain Length on Antibacterial Activity and Cell Selectivity. Molecules 2019, 24 (24).
32. Chen, Y.; Guarnieri, M. T.; Vasil, A. I.; Vasil, M. L.; Mant, C. T.; Hodges, R. S., Role of peptide hydrophobicity in the mechanism of action of alpha-helical antimicrobial peptides. Antimicrob Agents Chemother 2007, 51 (4), 1398-406.
33. Schroer M. A., S. D. I., Recent developments in small-angle X-ray scattering and hybrid method approaches for biomacromolecular solutions. Emerging Topics in Life Sciences 2018, 2 (1), 69-79.
34. Lund, R., Shu, J., Xu, T., A Small-Angle X-ray Scattering Study of α-helical Bundle-Forming Peptide-Polymer Conjugates in Solution: Chain Conformations. Macromolecules 2013, 46 (4), 1625-1632.
35. Narayanan T., W. H., Recent applications of synchrotron radiation and neutrons in the study of soft matter. Crystallography Reviews 2017, 23(3), 160-226.
36. Nielsen, J. E.; Bjornestad, V. A.; Lund, R., Resolving the structural interactions between antimicrobial peptides and lipid membranes using small-angle scattering methods: the case of indolicidin. Soft Matter 2018, 14 (43), 8750-8763.
37. bacteria, N. c. f. c. 1. s. m. f. d. a. s. t. f. b. t. g. a. a. s. n. e. M. f. d. a. s. t. f.
38. Vandecandelaere, I.; Van Nieuwerburgh, F.; Deforce, D.; Nelis, H. J.; Coenye, T., Draft Genome Sequence of Methicillin-Resistant Staphylococcus epidermidis Strain ET-024, Isolated from an Endotracheal Tube Biofilm of a Mechanically Ventilated Patient. Genome Announc 2014, 2 (3).
39. Pernot, P.; Round, A.; Barrett, R.; De Maria Antolinos, A.; Gobbo, A.; Gordon, E.; Huet, J.; Kieffer, J.; Lentini, M.; Mattenet, M.; Morawe, C.; Mueller-Dieckmann, C.; Ohlsson, S.; Schmid, W.; Surr, J.; Theveneau, P.; Zerrad, L.; McSweeney, S., Upgraded ESRF BM29 beamline for SAXS on macromolecules in solution. J Synchrotron Radiat 2013, 20 (Pt 4), 660-4.
40. Round, A.; Felisaz, F.; Fodinger, L.; Gobbo, A.; Huet, J.; Villard, C.; Blanchet, C. E.; Pernot, P.; McSweeney, S.; Roessle, M.; Svergun, D. I.; Cipriani, F., BioSAXS Sample Changer: a robotic sample changer for rapid and reliable high-throughput X-ray solution scattering experiments. Acta Crystallogr D Biol Crystallogr 2015, 71 (Pt 1), 67-75.
41. De Maria Antolinos, A.; Pernot, P.; Brennich, M. E.; Kieffer, J.; Bowler, M. W.; Delageniere, S.; Ohlsson, S.; Malbet Monaco, S.; Ashton, A.; Franke, D.; Svergun, D.; McSweeney, S.; Gordon, E.; Round, A., ISPyB for BioSAXS, the gateway to user autonomy in solution scattering experiments. Acta Crystallogr D Biol Crystallogr 2015, 71 (Pt 1), 76-85.
42. Mouritzen, M. V.; Abourayale, S.; Ejaz, R.; Ardon, C. B.; Carvalho, E.; Dalgaard, L. T.; Roursgaard, M.; Jenssen, H., Neurotensin, substance P, and insulin enhance cell migration. J Pept Sci 2018, 24 (7), e3093.
43. Nielsen, J. E.; Bjornestad, V. A.; Lund, R., Resolving the structural interactions between antimicrobial peptides and lipid membranes using small-angle scattering methods: the case of indolicidin. Soft Matter 2018, 14 (43), 8750-8763.
44. Schweizer, K. S., and J. G. Curro, PRISM theory of the structure, thermodynamics, and phase transitions of polymer liquids and alloys.” Atomistic Modeling of Physical Properties. Atomistic Modeling of Physical Properties. Springer, Berlin, Heidelberg 1994., 319-377.
45. Arleth L., B. M., Pedersen J. S., Small-angle neutron scattering study of the growth behavior, flexibility, and intermicellar interactions of wormlike SDS micelles in NaBr aqueous solutions. Langmuir. 2002, 18(14), 5343-53.

What is claimed is:

A1. A poly-N-substituted glycine compound having the formula 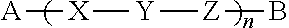
or a pharmaceutically acceptable salt thereof, wherein
A is a terminal N-alkyl substituted glycine residue;
n is an integer;
B is selected from the group consisting of NH₂, one and two N-substituted glycine residues, and wherein said one and two N-substituted glycine residues have N-substituents which are independently selected from natural α-amino acid side chain moieties, isomers and carbon homologs thereof; and
X, Y and Z are independently selected from the group consisting of N-substituted glycine residues, wherein said N-substituents are independently selected from the group consisting of natural α-amino acid side chain moieties, isomers and carbon homologs thereof, and proline residues, and wherein at least one of A, B, X, Y and Z contains a halogen-bearing moiety.

A2. The compound of claim A1, wherein said alkyl substituent is selected from about C₄ to about C₂₀ linear, branched and cyclic alkyl moieties.

A3. The compound of claim A1, wherein n has a value within the range of 1-3.

A4. The compound of claim A1, wherein at least one of said X, Y and Z residues is N_{Lys} and at least one said N-substituent is chiral.

A5. The compound of claim A1, wherein at least one of Y and Z are proline residues.

A6. The compound of claim A1, wherein Y and Z are proline residues.

A7. The compound of claim A1, wherein A is a terminal N-alkyl substituted glycine residue, wherein said alkyl substituent selected from the group consisting of C₆ to about C₁₈ linear alkyl moieties, wherein B is NH₂, and wherein n is 1 or 2.

A8. The compound of claim A1, wherein A is a terminal N-alkyl substituted glycine residue, said alkyl substituent selected from about C₆ to about C₁₈ linear alkyl moieties; wherein B is an N_{Lys} residue; and wherein n is 1.

A9. The compound of claim A1, wherein the compound is a hexamer.

A10. The compound of claim A1, wherein the compound is a dodecamer.

A11. The compound of claims A1-A10, wherein said halogen-bearing moiety contains a halogen-substituted aryl moiety.

A12. The compound of claims A1-A10, wherein said halogen-bearing moiety contains a chloro-substituted aryl moiety.

A13. The compound of claims A1-A10, wherein said halogen-bearing moiety contains a bromo-substituted aryl moiety.

A14. The compound of claims A1-A10, wherein said halogen-bearing moiety contains an iodo-substituted aryl moiety.

A15. The compound of claim A9, wherein each mer in the hexamer contains a halogen-substituted aryl moiety.

A16. The compound of claim A9, wherein some of the mers in the hexamer contain a halogen-substituted aryl moiety, and wherein some of the mers in the hexamer contain a halogen-free aryl moiety.

A17. The compound of claim A9, wherein exactly one of the mers in the hexamer contains a halogen-substituted aryl moiety.

A18. The compound of claim A10, wherein each mer in the hexamer contains a halogen-substituted aryl moiety.

A19. The compound of claim A10, wherein some of the mers in the hexamer contain a halogen-substituted aryl moiety, and wherein some of the mers in the hexamer contain a halogen-free aryl moiety.

A20. The compound of claim A10, wherein only the first and last mers in the hexamer contain a halogen-substituted aryl moiety.

A21. The compound of claim A1, wherein at least two of A, B, X, Y and Z contain a halogen-bearing moiety.

A22. The compound of claim A1, wherein all of A, B, X, Y and Z contain a halogen-bearing moiety.

A23. The compound of claim A1, wherein the compound is an antimicrobial compound.

A24. A pharmaceutically acceptable salt of the compound of claims A1-A23.

B1. A poly-N-alkyl substituted glycine compound of a formula 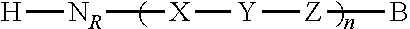
or a pharmaceutically acceptable salt thereof, wherein
B is selected from NH₂ and X′;
N_{R}, X, Y, Z and X′ are independently selected from N-substituted glycine residues containing N-substituents, wherein said N-substituents of said N-substituted glycine residues are independently selected from natural α-amino acid side chain moieties, isomers and carbon homologs thereof, and proline residues, and wherein at least one said N-substituent contains a halogen atom;
n is an integer; and
R is an N-alkyl substituent of said N_{R} glycine residue, said substituent selected from about C₄ to about C₂₀ linear, branched and cyclic alkyl moieties.

B2. The compound of claim B1, wherein n is 2 and B is NH₂.

B3. The compound of claim B1, wherein n is 1 and B is X′.

B4. The compound of claim B3 wherein at least one of X and X′ are N_{Lys} residues.

B5. The compound of claim B4, wherein said N-alkyl substituent is selected from about C₆ to about C₁₈ linear, branched and cyclic alkyl moieties.

B6. The compound of claim B5 wherein X and X′ are N_{Lys} residues.

B7. The compound of claim B6 of a formula

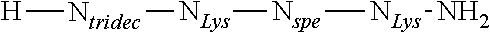

B8. The compound of claim B1, wherein said alkyl substituent is selected from about C₄ to about C₂₀ linear, branched and cyclic alkyl moieties.

B9. The compound of claim B1, wherein n has a value within the range of 1-2.

B10. The compound of claim B1, wherein at least one of said X, Y and Z residues is N_{Lys} and at least one said N-substituent is chiral.

B11. The compound of claim B1, wherein at least one of Y and Z are proline residues.

B12. The compound of claim B1, wherein Y and Z are proline residues.

B13. The compound of claim B1, wherein A is a terminal N-alkyl substituted glycine residue, wherein said alkyl substituent selected from the group consisting of C₆ to about C₁₈ linear alkyl moieties, wherein B is NH₂, and wherein n is 1 or 2.

B14. The compound of claim B1, wherein N_{R} is a terminal N-alkyl substituted glycine residue, said alkyl substituent selected from about C₆ to about C₁₈ linear alkyl moieties; wherein B is an N_{Lys} residue; and wherein n is 1.

B15. The compound of claim B1, wherein the compound is a hexamer.

B16. The compound of claim B1, wherein the compound is a dodecamer.

B17. The compound of claims B1-B16, wherein said halogen-bearing moiety contains a halogen-substituted aryl moiety.

B18. The compound of claims B1-B16, wherein said halogen-bearing moiety contains a chloro-substituted aryl moiety.

B19. The compound of claims B1-B16, wherein said halogen-bearing moiety contains a bromo-substituted aryl moiety.

B20. The compound of claims B1-B16, wherein said halogen-bearing moiety contains an iodo-substituted aryl moiety.

B21. The compound of claim B15, wherein each mer in the hexamer contains a halogen-substituted aryl moiety.

B22. The compound of claim B15, wherein some of the mers in the hexamer contain a halogen-substituted aryl moiety, and wherein some of the mers in the hexamer contain a halogen-free aryl moiety.

B23. The compound of claim B15, wherein exactly one of the mers in the hexamer contains a halogen-substituted aryl moiety.

B24. The compound of claim B16, wherein each mer in the hexamer contains a halogen-substituted aryl moiety.

B25. The compound of claim B16, wherein some of the mers in the hexamer contain a halogen-substituted aryl moiety, and wherein some of the mers in the hexamer contain a halogen-free aryl moiety.

B26. The compound of claim B16, wherein only the first and last mers in the hexamer contain a halogen-substituted aryl moiety.

B27. The compound of claim B1, wherein at least two of N_{R}, X, Y, Z and X′ contain a halogen-bearing moiety.

B28. The compound of claim B1, wherein all of N_{R}, X, Y, Z and X′ contain a halogen-bearing moiety.

B29. The compound of claim B1, wherein the compound is an antimicrobial compound.

B30. A pharmaceutically acceptable salt of the compound of claims B1-B29.

C1. A poly-N-substituted glycine compound or a pharmaceutically acceptable salt thereof, the compound comprising an N-terminal N-alkyl substituted glycine residue where said alkyl substituent is selected from about C₄ to about C₂₀ linear, branched and cyclic alkyl moieties; a C-terminus selected from NH₂, one and two N-substituted glycine residues, said N-substituents independently selected from α-amino acid side chain moieties and carbon homologs thereof, and 2 to about 15 monomeric residues between said N- and C-termini, each said residue independently selected from proline residues and N-substituted glycine residues, said N-substituents independently selected from natural α-amino acid side chain moieties, isomers and carbon homologs thereof, at least one said monomeric residue is N_{Lys} and at least one said N-substituent is chiral, said monomeric residues selected to provide said compound a non-periodic sequence of monomeric residues; and wherein at least one of said residues contains at least one halogen.

C2. The compound of claim C1, wherein said N-terminus is an N-alkyl substituted glycine residue, said alkyl substituent selected from about C₆ to about C₁₈ linear alkyl moieties.

C3. The compound of claim C2, wherein said monomeric residues comprise 2-5 (X—Y—Z) non-periodic trimers.

C4. The compound of claim C3, wherein at least one X, Y and Z in each of said trimers is selected to interrupt 3-fold periodicity.

C5. The compound of claim C3 wherein said monomeric residues comprise at least two non-consecutive repeat trimers, with at least one residue therebetween.

C6. The compound of claim C5, wherein at least one X in at least one said trimer is an N_{Lys} residue, and at least one of Y and Z in at least one said trimer is a proline residue.

C7. The compound of claim C1, wherein said at least one halogen is selected from the group consisting of bromine, chlorine and iodine.

C8. The compound of claim C1, wherein at least one of said residues contains at least one halogen-substituted aryl moiety.

C9. The compound of claim C1, wherein each of said residues contains at least one halogen-substituted aryl moiety.

C10. The compound of claim C1, wherein some of said residues contain at least one halogen-substituted aryl moiety, and wherein some of said residues contain at least one non-halogenated-substituted aryl moiety.

C11. The compound of claims C8-C10, wherein said halogen-substituted aryl moiety is para-substituted.

C12. The compound of claims C8-C10, wherein said halogen-substituted aryl moiety is perhalogenated.

C13. A pharmaceutically acceptable salt of the compound of claims C1-C12.

D1. A compound derived from a material selected from the group consisting of the compound of FIG. 1 and the compound of FIG. 13 by substituting at least one hydrogen atom in at least one aryl moiety thereof with at least one halogen atom.

D2. The compound of claim D1, wherein said compound is derived from a material selected from the group consisting of the compound of FIG. 1 and the compound of FIG. 13 by substituting at least one para-hydrogen atom in at least one aryl moiety thereof with at least one halogen atom.

D3. The compound of claim D1, wherein said compound is derived from a material selected from the group consisting of the compound of FIG. 1 and the compound of FIG. 13 by substituting one hydrogen atom in each aryl moiety thereof with at least one halogen atom.

D4. The compound of claims D1-D3, wherein said at least one halogen atom is selected from the group consisting of chlorine, bromine and iodine.

D5. The compound of claims D1-D3, wherein said at least one halogen atom includes at least first and second distinct halogens selected from the group consisting of chlorine, bromine and iodine.

D6. The compound of claim D1, wherein said compound is

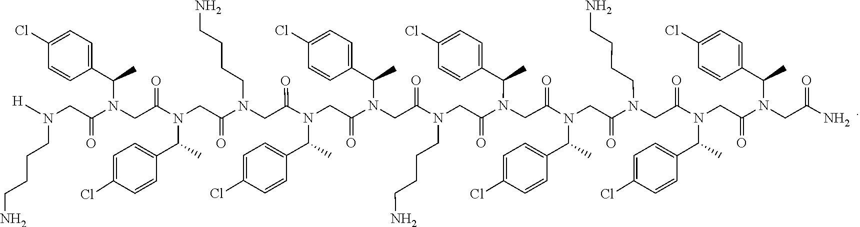 [SMILES: C[C@H](c1ccc(Cl)cc1)N(CC(N)=O)C(=O)CN(C(=O)CN(CCCCN)C(=O)CN(C(=O)CN(C(=O)CN(CCCCN)C(=O)CN(C(=O)CN(C(=O)CN(CCCCN)C(=O)CN(C(=O)CN(C(=O)CNCCCCN)[C@H](C)c1ccc(Cl)cc1)[C@H](C)c1ccc(Cl)cc1)[C@H](C)c1ccc(Cl)cc1)[C@H](C)c1ccc(Cl)cc1)[C@H](C)c1ccc(Cl)cc1)[C@H](C)c1ccc(Cl)cc1)[C@H](C)c1ccc(Cl)cc1]

D7. The compound of claim D1, wherein said compound is

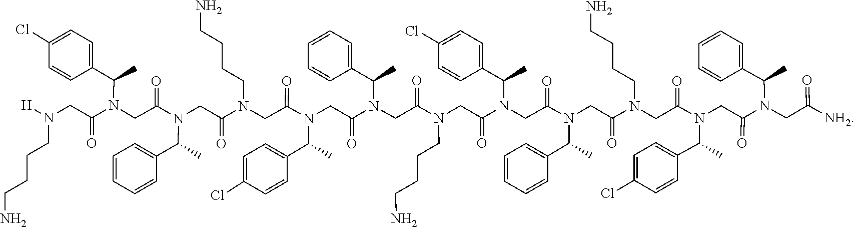 [SMILES: C[C@H](c1ccccc1)N(CC(N)=O)C(=O)CN(C(=O)CN(CCCCN)C(=O)CN(C(=O)CN(C(=O)CN(CCCCN)C(=O)CN(C(=O)CN(C(=O)CN(CCCCN)C(=O)CN(C(=O)CN(C(=O)CNCCCCN)[C@H](C)c1ccc(Cl)cc1)[C@H](C)c1ccccc1)[C@H](C)c1ccc(Cl)cc1)[C@H](C)c1ccccc1)[C@H](C)c1ccc(Cl)cc1)[C@H](C)c1ccccc1)[C@H](C)c1ccc(Cl)cc1]

D8. The compound of claim D1, wherein said compound is

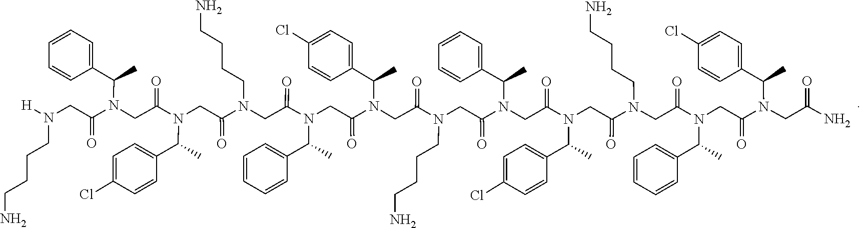 [SMILES: C[C@H](c1ccc(Cl)cc1)N(CC(N)=O)C(=O)CN(C(=O)CN(CCCCN)C(=O)CN(C(=O)CN(C(=O)CN(CCCCN)C(=O)CN(C(=O)CN(C(=O)CN(CCCCN)C(=O)CN(C(=O)CN(C(=O)CNCCCCN)[C@H](C)c1ccccc1)[C@H](C)c1ccc(Cl)cc1)[C@H](C)c1ccccc1)[C@H](C)c1ccc(Cl)cc1)[C@H](C)c1ccccc1)[C@H](C)c1ccc(Cl)cc1)[C@H](C)c1ccccc1]

D9. A pharmaceutically acceptable salt of the compound of claims D1-D8.

E1. A method for treating or inhibiting a disease, comprising administering to an individual who has, or is at risk of developing said disease, an amount of at least one poly-N-alkyl substituted glycine or a pharmaceutically acceptable salt thereof, wherein the amount of the poly-N-alkyl substituted glycine is effective to treat or inhibit said disease, and wherein the poly-N-alkyl substituted glycine compound has the formula 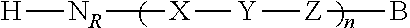
wherein
B is selected from NH₂ and X′;
N_{R}, X, Y, Z and X′ are independently selected from N-substituted glycine residues containing N-substituents, wherein said N-substituents of said N-substituted glycine residues are independently selected from natural α-amino acid side chain moieties, isomers and carbon homologs thereof, and proline residues, and wherein at least one said N-substituent contains a halogen atom;
n is an integer; and
R is an N-alkyl substituent of said N_{R} glycine residue, said substituent selected from about C₄ to about C₂₀ linear, branched and cyclic alkyl moieties.

E2. The method of claim E1, wherein n is 2 and B is NH₂.

E3. The method of claim E1, wherein n is 1 and B is X′.

E4. The method of claim E3 wherein at least one of X and X′ are N_{Lys} residues.

E5. The method of claim E4, wherein said N-alkyl substituent is selected from about C₆ to about C₁₈ linear, branched and cyclic alkyl moieties.

E6. The method of claim E5 wherein X and X′ are N_{Lys} residues.

E7. The method of claim E6 of a formula
H—N_{tridec}—N_{Lys}—Nₛₚₑ—Nₛₚₑ—N_{Lys}—NH₂.

E8. The method of claim E1, wherein said alkyl substituent is selected from about C₄ to about C₂₀ linear, branched and cyclic alkyl moieties.

E9. The method of claim E1, wherein n has a value within the range of 1-2.

E10. The method of claim E1, wherein at least one of said X, Y and Z residues is N_{Lys} and at least one said N-substituent is chiral.

E11. The method of claim E1, wherein at least one of Y and Z are proline residues.

E12. The method of claim E1, wherein Y and Z are proline residues.

E13. The method of claim E1, wherein A is a terminal N-alkyl substituted glycine residue, wherein said alkyl substituent selected from the group consisting of C₆ to about C₁₈ linear alkyl moieties, wherein B is NH₂, and wherein n is 1 or 2.

E14. The method of claim E1, wherein N_{R} is a terminal N-alkyl substituted glycine residue, said alkyl substituent selected from about C₆ to about C₁₈ linear alkyl moieties; wherein B is an N_{Lys} residue; and wherein n is 1.

E15. The method of claim E1, wherein the compound is a hexamer.

E16. The method of claim E1, wherein the compound is a dodecamer.

E17. The method of claims E1-E16, wherein said halogen-bearing moiety contains a halogen-substituted aryl moiety.

E18. The method of claims E1-E16, wherein said halogen-bearing moiety contains a chloro-substituted aryl moiety.

E19. The method of claims E1-E16, wherein said halogen-bearing moiety contains a bromo-substituted aryl moiety.

E20. The method of claims E1-E16, wherein said halogen-bearing moiety contains an iodo-substituted aryl moiety.

E21. The method of claim E15, wherein each mer in the hexamer contains a halogen-substituted aryl moiety.

E22. The method of claim E15, wherein some of the mers in the hexamer contain a halogen-substituted aryl moiety, and wherein some of the mers in the hexamer contain a halogen-free aryl moiety.

E23. The method of claim E15, wherein exactly one of the mers in the hexamer contains a halogen-substituted aryl moiety.

E24. The method of claim E16, wherein each mer in the hexamer contains a halogen-substituted aryl moiety.

E25. The method of claim E16, wherein some of the mers in the hexamer contain a halogen-substituted aryl moiety, and wherein some of the mers in the hexamer contain a halogen-free aryl moiety.

E26. The method of claim E16, wherein only the first and last mers in the hexamer contain a halogen-substituted aryl moiety.

E27. The method of claim E1, wherein at least two of N_{R}, X, Y, Z and X′ contain a halogen-bearing moiety.

E28. The method of claim E1, wherein all of N_{R}, X, Y, Z and X′ contain a halogen-bearing moiety.

E29. The method of claim E1, wherein the compound is an antimicrobial compound.

E30. The method of claim E1, wherein the disease is caused by an enveloped RNA virus.

E31. The method of claim E1, wherein the disease is caused by a coronavirus.

E32. The method of claim E31, wherein the disease is selected from the group consisting of severe acute respiratory syndrome (SARS), Middle East respiratory syndrome (MERS) and coronavirus disease 19 (COVID-19).

E33. The method of claim E31, wherein the disease is COVID-19.

F1. A method for treating or inhibiting a disease, comprising administering to an individual who has, or is at risk of developing said disease, an amount of at least one poly-N-alkyl substituted glycine or a pharmaceutically acceptable salt thereof, wherein the amount of the poly-N-alkyl substituted glycine is effective to treat or inhibit said disease, and wherein the poly-N-alkyl substituted glycine compound is a compound, or a pharmaceutically acceptable salt thereof, which is derived from a material selected from the group consisting of the compound of FIG. 1 and the compound of FIG. 13 by substituting at least one hydrogen atom in at least one aryl moiety thereof with at least one halogen atom.

F2. The method of claim F1, wherein said compound is derived from a material selected from the group consisting of the compound of FIG. 1 and the compound of FIG. 13 by substituting at least one para-hydrogen atom in at least one aryl moiety thereof with at least one halogen atom.

F3. The method of claim F1, wherein said compound is derived from a material selected from the group consisting of the compound of FIG. 1 and the compound of FIG. 13 by substituting one hydrogen atom in each aryl moiety thereof with at least one halogen atom.

F4. The method of claims F1-F3, wherein said at least one halogen atom is selected from the group consisting of chlorine, bromine and iodine.

F5. The method of claims F1-F3, wherein said at least one halogen atom includes at least first and second distinct halogens selected from the group consisting of chlorine, bromine and iodine.

F6. The method of claim F1, wherein said compound is

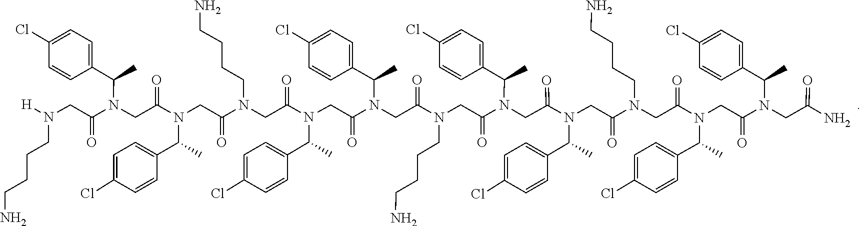 [SMILES: C[C@H](c1ccc(Cl)cc1)N(CC(N)=O)C(=O)CN(C(=O)CN(CCCCN)C(=O)CN(C(=O)CN(C(=O)CN(CCCCN)C(=O)CN(C(=O)CN(C(=O)CN(CCCCN)C(=O)CN(C(=O)CN(C(=O)CNCCCCN)[C@H](C)c1ccc(Cl)cc1)[C@H](C)c1ccc(Cl)cc1)[C@H](C)c1ccc(Cl)cc1)[C@H](C)c1ccc(Cl)cc1)[C@H](C)c1ccc(Cl)cc1)[C@H](C)c1ccc(Cl)cc1)[C@H](C)c1ccc(Cl)cc1]

F7. The method of claim F1, wherein said compound is

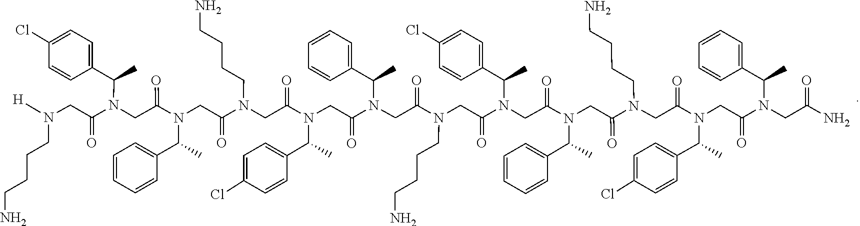 [SMILES: C[C@H](c1ccccc1)N(CC(N)=O)C(=O)CN(C(=O)CN(CCCCN)C(=O)CN(C(=O)CN(C(=O)CN(CCCCN)C(=O)CN(C(=O)CN(C(=O)CN(CCCCN)C(=O)CN(C(=O)CN(C(=O)CNCCCCN)[C@H](C)c1ccc(Cl)cc1)[C@H](C)c1ccccc1)[C@H](C)c1ccc(Cl)cc1)[C@H](C)c1ccccc1)[C@H](C)c1ccc(Cl)cc1)[C@H](C)c1ccccc1)[C@H](C)c1ccc(Cl)cc1]

F8. The method of claim F1, wherein said compound is

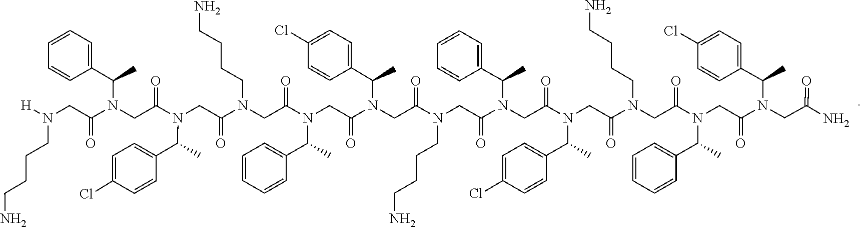 [SMILES: C[C@H](c1ccc(Cl)cc1)N(CC(N)=O)C(=O)CN(C(=O)CN(CCCCN)C(=O)CN(C(=O)CN(C(=O)CN(CCCCN)C(=O)CN(C(=O)CN(C(=O)CN(CCCCN)C(=O)CN(C(=O)CN(C(=O)CNCCCCN)[C@H](C)c1ccccc1)[C@H](C)c1ccc(Cl)cc1)[C@H](C)c1ccccc1)[C@H](C)c1ccc(Cl)cc1)[C@H](C)c1ccccc1)[C@H](C)c1ccc(Cl)cc1)[C@H](C)c1ccccc1]

F9. The method of claim F1, wherein the disease is caused by an enveloped RNA virus.

F10. The method of claim F1, wherein the disease is caused by a coronavirus.

F11. The method of claim F10, wherein the disease is selected from the group consisting of severe acute respiratory syndrome (SARS), Middle East respiratory syndrome (MERS) and coronavirus disease 19 (COVID-19).

F12. The method of claim F10, wherein the disease is COVID-19.

G1. A method for treating or inhibiting a disease, comprising administering to an individual who has, or is at risk of developing said disease, an amount of at least one poly-N-alkyl substituted glycine or a pharmaceutically acceptable salt thereof, wherein the amount of the poly-N-alkyl substituted glycine is effective to treat or inhibit said disease, and wherein the poly-N-alkyl substituted glycine compound comprises an N-terminal N-alkyl substituted glycine residue where said alkyl substituent is selected from about C₄ to about C₂₀ linear, branched and cyclic alkyl moieties; a C-terminus selected from NH₂, one and two N-substituted glycine residues, said N-substituents independently selected from α-amino acid side chain moieties and carbon homologs thereof; and 2 to about 15 monomeric residues between said N- and C-termini, each said residue independently selected from proline residues and N-substituted glycine residues, said N-substituents independently selected from natural α-amino acid side chain moieties, isomers and carbon homologs thereof, at least one said monomeric residue is N_{Lys} and at least one said N-substituent is chiral, said monomeric residues selected to provide said compound a non-periodic sequence of monomeric residues; and wherein at least one of said residues contains at least one halogen.

G2. The method of claim G1, wherein said N-terminus is an N-alkyl substituted glycine residue, said alkyl substituent selected from about C₆ to about C₁₈ linear alkyl moieties.

G3. The method of claim G2, wherein said monomeric residues comprise 2-5 (X—Y—Z) non-periodic trimers.

G4. The method of claim G3, wherein at least one X, Y and Z in each of said trimers is selected to interrupt 3-fold periodicity.

G5. The method of claim G3 wherein said monomeric residues comprise at least two non-consecutive repeat trimers, with at least one residue therebetween.

G6. The method of claim G5, wherein at least one X in at least one said trimer is an N_{Lys} residue, and at least one of Y and Z in at least one said trimer is a proline residue.

G7. The method of claim G1, wherein said at least one halogen is selected from the group consisting of bromine, chlorine and iodine.

G8. The method of claim G1, wherein at least one of said residues contains at least one halogen-substituted aryl moiety.

G9. The method of claim G1, wherein each of said residues contains at least one halogen-substituted aryl moiety.

G10. The method of claim G1, wherein some of said residues contain at least one halogen-substituted aryl moiety, and wherein some of said residues contain at least one non-halogenated-substituted aryl moiety.

G11. The method of claims G8-G10, wherein said halogen-substituted aryl moiety is para-substituted.

G12. The method of claims C8-C10, wherein said halogen-substituted aryl moiety is perhalogenated.

G13. The method of claims G1-G12, wherein the poly-N-alkyl substituted glycine is a pharmaceutically acceptable salt of the compound of claims C1-C12.

G14. The method of claim G1, wherein the disease is caused by an enveloped RNA virus.

G15. The method of claim G1, wherein the disease is caused by a coronavirus.

G16. The method of claim G15, wherein the disease is selected from the group consisting of severe acute respiratory syndrome (SARS), Middle East respiratory syndrome (MERS) and coronavirus disease 19 (COVID-19).

G17. The method of claim G15, wherein the disease is COVID-19.

H1. A poly-N-substituted glycine compound containing at least one halogen selected from the group consisting of chlorine, bromine and iodine.

H2. The poly-N-substituted glycine compound of claim H1, wherein the poly-N-substituted glycine compound contains at least two halogens selected from the group consisting of chlorine, bromine and iodine.

H3. The method of claim H2, wherein the poly-N-substituted glycine compound contains at least two bromine atoms.

H4. The method of claim H2, wherein the poly-N-substituted glycine compound contains at least two chlorine atoms.

H5. The method of claim H2, wherein the poly-N-substituted glycine compound contains at least four bromine atoms.

H6. The method of claim H2, wherein the poly-N-substituted glycine compound contains at least four chlorine atoms.

H7. The method of claim H2, wherein the poly-N-substituted glycine compound contains at least one parahalogenated aryl group.

H8. The method of claim H1, wherein the poly-N-substituted glycine compound contains at least two parahalogenated aryl groups.

H9. The method of claim H1, wherein the poly-N-substituted glycine compound contains at least four parahalogenated aryl groups.

H10. The method of claim H2, wherein the poly-N-substituted glycine compound contains at least one parachlorinated aryl group.

H11. The method of claim H1, wherein the poly-N-substituted glycine compound contains at least two parachlorinated aryl groups.

H12. The method of claim H1, wherein the poly-N-substituted glycine compound contains at least four parachlorinated aryl groups.

H13. The method of claim H1, wherein the poly-N-substituted glycine compound contains at least one parabrominated aryl group.

H14. The method of claim H1, wherein the poly-N-substituted glycine compound contains at least two parabrominated aryl groups.

H15. The method of claim H1, wherein the poly-N-substituted glycine compound contains at least four parabrominated aryl groups.

H16. The method of claim H1, wherein the poly-N-substituted glycine compound contains a carboxamide terminus group.

H17. The method of claim H1, wherein the poly-N-substituted glycine compound is made via a rink amide resin.
